SEQUENCE LISTING (4 sequences)

SEQ ID NO: 1 - protein, 39 aa, synthetic construct
  REGION 1..39: note = Synthetic Construct
  SITE 2: note = X at position 2 is Aib
  SITE 13: note = X at position 13 is Aib
  MOD_RES 20: note = Lys at position 20 is chemically modified through conjugation to the epsilon-amino group of the K side-chain with (2-[2-(2-Amino-ethoxy)-ethoxy]-acetyl)2-(gamma-Glu)1- CO-(CH2)18-CO2H
  MOD_RES 39: note = C-terminal amino acid is amidated as a C-terminal primary amide
YXEGTFTSDYSIXLDKIAQKAFVQWLIAGGPSSGAPPPS

SEQ ID NO: 2 - protein, 39 aa, synthetic construct
  SITE 2: note = X at position 2 is Aib
  SITE 13: note = X at position 13 is Aib
  MOD_RES 20: note = K at position 20 is chemically modified through conjugation to the epsilon-amino group of the K side-chain with a C16-C20 fatty acid or a derivative thereof
  MOD_RES 39: note = C-terminal amino acid is optionally amidated as a C-terminal primary amide
  SITE 22: note = X at position 22 is Phe
YXEGTFTSDYSIXLDKIAQKAXVQWLIAGGPSSGAPPPS

SEQ ID NO: 3 - protein, 39 aa, synthetic construct; identical to SEQ ID NO: 1
  SITE 2: note = X at position 2 is Aib
  SITE 13: note = X at position 13 is Aib
  MOD_RES 20: note = K at position 20 is chemically modified through conjugation to the epsilon-amino group of the K side-chain with a fatty acid selected from the group consisting of (2-[2-(2-Amino-ethoxy)-ethoxy]-acetyl)2-(gamma-Glu)1-CO-(C H2)18-PO(OH)2, (2-[2-(2-Amino-ethoxy)-ethoxy]-acetyl)2-CH2 CH2-CHPO(OH)2-CO-(CH2)18-CO2H, and (2-[2-(2-Amino-ethoxy)- ethoxy]-acetyl)2-CO-CH2CH2-CHPO(OH)2-CO-(CH2)18-PO(OH)2
  MOD_RES 39: note = C-terminal amino acid is optionally amidated as a C-terminal primary amide

SEQ ID NO: 4 - protein, 39 aa, synthetic construct; identical to SEQ ID NO: 1
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)-(Aib)-(gamma-G lu)-CO-(CH2)18-CO2H
  MOD_RES 39: note = The C-terminal amino acid is amidated as a C-terminal primary amide

(19) United States
(12) Patent Application Publication
(10) Pub. No.: US 2026/0248891 A1
(43) Pub. Date: Aug. 27, 2026

(54) TIRZEPATIDE COMPOSITIONS AND USE

(71) Applicant: Eli Lilly and Company, Indianapolis, IN (US)

(72) Inventors: Michael J. KOENIG, Fishers, IN (US); Ken Kangyi QIAN, Carmel, IN (US)

(21) Appl. No.: 18/878,123

(22) PCT Filed: Jun. 23, 2023
(86) PCT No.: PCT/US2023/068925

§ 371 Date: Dec. 23, 2024

Related U.S. Application Data
(60) Provisional application No. 63/357,285, filed on Jun. 30, 2022.

(51) Int. Cl. A61K 38/26; A61K 9/00; A61K 47/02; A61P 3/04; A61P 3/10

(52) U.S. Cl. CPC A61K 38/26; A61K 9/0019; A61K 47/02; A61P 3/04; A61P 3/10

(57) ABSTRACT

A composition and methods of dosing such tirzepatide composition, comprising a tirzepatide, or a salt thereof at a concentration selected from the group consisting of 2.5 mg/mL, 40 mg/mL, and 50 mg/mL; NaCl; and dibasic sodium phosphate is provided.

The present application is being filed along with a Sequence Listing in ST.26 XML format. The Sequence Listing is provided as a file titled “30144_US.xml” created Dec. 11, 2024, and is 12.1 kilobytes in size. The Sequence Listing information in the ST.26 XML format is incorporated herein by reference in its entirety. No new matter is added herewith.

The present invention relates to the field of medicine. More particularly, the present invention relates to methods of using new doses of tirzepatide and pharmaceutically elegant compositions comprising such doses. The compositions provide commercially acceptable shelf-life stability, in-use stability, and are associated with acceptable patient experience.

Diabetes mellitus is a chronic disorder characterized by hyperglycemia resulting from defects in insulin secretion, insulin action, or both. In type 2 diabetes mellitus (“T2D”), the combined effects of impaired insulin secretion and insulin resistance are associated with elevated blood glucose levels.

Tirzepatide, the active ingredient in Mounjaro™ is a glucose-dependent insulinotropic polypeptide (GIP) receptor and glucagon-like peptide1 (GLP1) receptor agonist (GIP/GLP1) approved for use as an adjunct to diet and exercise to improve glycemic control in patients with T2D. Tirzepatide is approved as a clear colorless to slightly yellow liquid for subcutaneous injection. Once weekly tirzepatide doses of 2.5 mg, 5 mg, 7.5 mg, 10 mg, 12.5 mg, and 15 mg are administered using a starting dose 2.5 mg once weekly; after 4 weeks, increase to 5 mg injected subcutaneously once weekly; increasing the dosage in 2.5 mg increments after at least 4 weeks on the current dose. The maximum approved dosage is 15 mg subcutaneously once weekly. Tirzepatide was studied in Phase 3 development programs and the once weekly doses received regulatory approval in the United States (US), European Union and other jurisdictions in 2022 for use in treating T2D. Since their approval in 2022, these doses of tirzepatide have been used to treat many patients with T2D. These doses have been studied in thousands of patients for use in chronic weight management. While therapy with currently approved tirzepatide enabled substantially all patients included in the Phase 3 programs to attain their glycemic targets (with or without use of other concomitant medications for T2D) and patients with obesity or overweight in a Phase 3 study achieved 15.7% weight loss, a significant number of patients receiving approved therapies require additional chronic weight management to achieve their weight management treatment goals. A significant number of patients receiving approved therapies require additional weight management benefit. Such patients are unable to achieve their weight management goals using currently approved pharmaceutical treatments and/or dosages of agents that enable weight loss. Gastrointestinal adverse events have been reported to limit higher doses of agents having GLP1 receptor agonist activity. The US label for tirzepatide (May 2022) states that use of tirzepatide may be associated with gastrointestinal adverse reaction, sometimes severe. There is a need for a dosing regimen to enable administration of higher tirzepatide doses with acceptable patient experience. There is an important medical need for pharmaceutical agents to enable additional weight management, while preserving an overall acceptable risk/benefit profile and patient tolerability.

There is a need for pharmaceutical compositions with higher doses of tirzepatide that are stable for storage for at least 6 months.

Peptides have unique physiochemical properties that often present challenges for achieving stable, pharmaceutically elegant compositions. For example, self-association, aggregation, adsorption to surfaces, solubility, and chemical stability of peptides present unique challenges for enabling the desired peptide composition. Bak, et al. “Physicochemical and Formulation Developability Assessment for Therapeutic Peptide Delivery—A Primer—PMC (nih.gov)” AAPS J. 2015 January; 17(1): 144-155.

The compositions, doses, and methods provide these benefits while maintaining an acceptable profile of safety risks and adverse events.

The present invention provides a pharmaceutically-acceptable composition of tirzepatide, or a pharmaceutically acceptable salt thereof; comprising; greater than 30 mg/mL tirzepatide or a pharmaceutically acceptable salt thereof, a tonicity agent, and sodium phosphate.

In an embodiment, is a composition of tirzepatide or a pharmaceutically acceptable salt thereof, comprising greater than 30 mg/mL tirzepatide, or a pharmaceutically acceptable salt thereof, a tonicity agent, and dibasic sodium phosphate.

In an embodiment, is a composition of tirzepatide or a pharmaceutically acceptable salt thereof, comprising greater than 30 mg/mL, and up to 60 mg/mL, tirzepatide, or a pharmaceutically acceptable salt thereof, a tonicity agent, and dibasic sodium phosphate.

In an embodiment, a tonicity agent is NaCl. In an embodiment, the NaCl concentration is from about 7 mg/mL to about 9 mg/mL. In an embodiment the NaCl concentration is from about 7.4 mg/mL to about 9.0 mg/mL. In an embodiment, the NaCl concentration is about 8.2 mg/mL.

In an embodiment, the dibasic sodium phosphate concentration is from about 0.7 mg/mL to about 1.5 mg/mL. In an embodiment, the dibasic sodium phosphate concentration is about 1.34 mg/mL.

In an embodiment, tirzepatide, or salt thereof, concentration is from about 40 mg/mL to about 50 mg/mL. In an embodiment, tirzepatide, or a pharmaceutically acceptable salt thereof, concentration is about 40 mg/mL. In an embodiment, tirzepatide, or a salt thereof, concentration is about 50 mg/mL. In an embodiment, tirzepatide, or a salt thereof, concentration is about 2.5 mg/mL. In an embodiment, tirzepatide, or a pharmaceutically acceptable salt thereof, concentration is from 40 mg/mL to 50 mg/mL. In an embodiment, tirzepatide, or a pharmaceutically acceptable salt thereof, concentration is 40 mg/mL. In an embodiment, tirzepatide, or a pharmaceutically acceptable salt thereof, concentration is 50 mg/mL. In an embodiment, tirzepatide, or a pharmaceutically acceptable salt thereof concentration is 2.5 mg/mL.

In an embodiment, tirzepatide, or a pharmaceutically acceptable salt thereof, concentration is from about 40 mg/mL to about 60 mg/mL. In an embodiment, tirzepatide, or a pharmaceutically acceptable salt thereof concentration is about 55 mg/mL. In an embodiment, tirzepatide, or a pharmaceutically acceptable salt thereof concentration is about 60 mg/mL. In an embodiment, tirzepatide, or a pharmaceutically acceptable salt thereof, concentration is 60 mg/mL. In an embodiment, tirzepatide, or a pharmaceutically acceptable salt thereof, concentration is 55 mg/mL. In an embodiment, or a pharmaceutically acceptable salt thereof, concentration is from 40 mg/mL to 60 mg/mL. In an embodiment, tirzepatide, or a pharmaceutically acceptable salt thereof, is tirzepatide as a free base.

In an embodiment, tirzepatide, or salt thereof, concentration is from about 40 mg/mL to about 50 mg/mL; NaCl concentration is about 8.2 mg/mL and dibasic sodium phosphate concentration is about 1.34 mg/mL. In an embodiment, tirzepatide, or salt thereof, concentration is about 2.5 mg/mL; NaCl concentration is about 8.2 mg/mL, dibasic sodium phosphate concentration is from about 1.2 mg/mL to about 1.34 mg/mL. In an embodiment, tirzepatide, or a pharmaceutically acceptable salt thereof, concentration is about 40 mg/mL; NaCl concentration is about 8.2 mg/mL, dibasic sodium phosphate concentration is about 1.34 mg/mL. In an embodiment, tirzepatide, or a pharmaceutically acceptable salt thereof, concentration is about 50 mg/mL; NaCl concentration is about 8.2 mg/mL, dibasic sodium phosphate concentration is about 1.34 mg/mL. In an embodiment, tirzepatide, or a pharmaceutically acceptable salt thereof, concentration is about 2.5 mg/mL; NaCl concentration is about 8.2 mg/mL, dibasic sodium phosphate concentration is from about 1.34 mg/mL. In an embodiment, tirzepatide concentration is about 40 mg/mL; NaCl concentration is about 8.2 mg/mL, dibasic sodium phosphate concentration is about 1.34 mg/mL, and the composition is presented in a single use automatic injection apparatus. In an embodiment, tirzepatide concentration is about 50 mg/mL; NaCl concentration is about 8.2 mg/mL, dibasic sodium phosphate concentration is about 1.34 mg/mL, and the composition is presented in a single use automatic injection apparatus. In an embodiment, tirzepatide concentration is about 2.5 mg/mL; NaCl concentration is about 8.2 mg/mL, dibasic sodium phosphate concentration is from about 1.34 mg/mL, and the composition is presented in a single use automatic injection apparatus.

In an embodiment tirzepatide, or a pharmaceutically acceptable salt thereof, concentration in a composition is selected from the group consisting of 2.5, 40, and 50 mg/mL. In an embodiment, tirzepatide or a pharmaceutically acceptable salt thereof is administered as a 0.5 mL dose. In an embodiment, a composition comprises tirzepatide, or a pharmaceutically acceptable salt thereof, formulated to deliver 1.25 mg/dose. In an embodiment, a tirzepatide, or pharmaceutically acceptable salt thereof, concentration is selected from the group consisting of 1.25 mg/0.5 mL, 20 mg/0.5 mL, and 25 mg/0.5 mL. In an embodiment tirzepatide, or a pharmaceutically acceptable salt thereof concentration is 30 mg/0.5 mL. In an embodiment, concentration of tirzepatide, or a pharmaceutically acceptable salt thereof, is the concentration of tirzepatide as a free base.

In an embodiment, is a method of treating chronic obesity in a subject in need thereof, comprising administering 1.25 mg tirzepatide, or a pharmaceutically acceptable salt thereof, for a minimum of four weeks.

In an embodiment, is a method of treating chronic obesity in a pediatric subject in need thereof, comprising administering 1.25 mg tirzepatide, or a pharmaceutically acceptable salt thereof, for a minimum of four weeks.

In an embodiment, is a method of treating type 2 diabetes in a subject in need thereof, comprising administering 1.25 mg tirzepatide, or a pharmaceutically acceptable salt thereof, for a minimum of four weeks.

In an embodiment, is a method of treating type 2 diabetes in a pediatric subject in need thereof, comprising administering 1.25 mg tirzepatide, or a pharmaceutically acceptable salt thereof, for a minimum of four weeks.

In an embodiment, is a method for chronic weight management in a pediatric subject comprising administering 1.25 mg tirzepatide, or a pharmaceutically acceptable salt thereof, to a subject in need thereof for a minimum of four weeks.

In an embodiment is a method for chronic weight management in a subject in need of additional weight management; wherein the subject has obesity, comprising:
identify a subject with obesity and in need of additional weight management;
administer to said subject a once weekly dose of tirzepatide, or a pharmaceutically
acceptable salt thereof, that is at least 15 mg for a minimum of four weeks; and
after at least four weeks administering the at least 15 mg once weekly dose of tirzepatide, or a pharmaceutically acceptable salt thereof, administer a once weekly dose of tirzepatide, or a pharmaceutically acceptable salt thereof, that is 5 mg greater than the last dose; wherein the maximum once weekly tirzepatide, or a pharmaceutically acceptable salt thereof, once weekly dose is 25 mg.

The present invention provides a method for chronic weight management in a subject in need of additional weight management, comprising: identifying a subject desiring weight management, and in need of additional weight management; comprising
a) administering to said subject a dose of tirzepatide, or a pharmaceutically acceptable salt thereof, that is >15 mg and <20 mg once weekly for a minimum of four weeks; and
   increasing the dose to 20 mg once weekly.

Accordingly, the present invention provides a method of chronic weight management in a subject in need of additional weight management, comprising:
a) identifying a subject in need of additional weight management;
b) administering to said subject 20 mg dose of tirzepatide, or a pharmaceutically acceptable salt thereof, once weekly for a minimum of four weeks; and
c) increasing the dose by administering to said subject 25 mg dose of tirzepatide, or a pharmaceutically acceptable salt thereof, once weekly.

Provided is a method of chronic weight management in a subject in need of additional weight management, comprising:
a) identifying a subject desiring weight management, and in need of additional weight management;
b) administering to said subject 20 mg dose of tirzepatide, or a pharmaceutically acceptable salt thereof, once weekly for a minimum of four weeks; and
c) Increasing the dose by administering to said subject 25 mg dose of tirzepatide, or a pharmaceutically acceptable salt thereof, once weekly.

In an aspect, provided is a method of chronic weight management in a subject in need of additional weight management, comprising:
a) identifying a subject with an initial BMI≥30 kg/m² and in need of additional weight management;
b) increasing the dose administering to said subject 20 mg dose of tirzepatide, or a pharmaceutically acceptable salt thereof, once weekly.

In another aspect, the present invention provides a method of providing chronic weight management to a subject in need thereof, comprising:
a) identifying a subject previously treated with at least 15 mg of tirzepatide, or a pharmaceutically acceptable salt thereof, once weekly for a minimum of four weeks; and
b) increasing the dose by administering 20 mg tirzepatide, or a pharmaceutically acceptable salt thereof, once weekly.

In another aspect, the present invention provides a method of providing chronic weight management to a subject in need thereof, comprising:
a) identifying a subject previously treated with at least 20 mg of tirzepatide, or a pharmaceutically acceptable salt thereof, once weekly for a minimum of four weeks; and
b) increasing the dose by administering 25 mg tirzepatide, or a pharmaceutically acceptable salt thereof, once weekly.

In another aspect, the present invention provides use of tirzepatide for the manufacture of a medicament for improving glycemic control in a subject having type 2 diabetes (T2D) and being treated with a 15 mg dose of tirzepatide once weekly but in need of additional glycemic control, comprising: increasing the dose of tirzepatide being administered to 20 mg once weekly.

In another aspect, the present invention provides use of tirzepatide for the manufacture of a medicament for improving glycemic control in a subject having type 2 diabetes (T2D) and being treated with a 20 mg dose of tirzepatide once weekly for at least four weeks, but in need of additional glycemic control, comprising: increasing the dose of tirzepatide being administered to 25 mg once weekly.

In another aspect, the present invention provides use of tirzepatide for the manufacture of a medicament for providing chronic weight management to a subject in need thereof, comprising:
a) administering to said subject 15 mg of tirzepatide once weekly for a minimum of four weeks;
b) Increasing the dose by administering to said subject 20 mg of tirzepatide once weekly for a minimum of four weeks; and
c) increasing the dose by administering 25 mg of tirzepatide once weekly.

In another aspect, the present invention provides use of tirzepatide for the manufacture of a medicament for providing chronic weight management to a subject in need thereof, comprising:
a) identifying a subject who has been previously treated with 15 mg of tirzepatide, or a pharmaceutically acceptable salt thereof, once weekly for a minimum of four weeks; and
b) increasing the dose by administering 20 mg tirzepatide, or a pharmaceutically acceptable salt thereof, once weekly.

In another aspect, the present invention provides use of tirzepatide for the manufacture of a medicament for providing chronic weight management to a subject in need thereof, comprising:
a) identifying a subject who has been previously treated with 20 mg of tirzepatide once weekly for a minimum of four weeks; and
b) increasing the dose by administering 25 mg tirzepatide, or a pharmaceutically acceptable salt thereof, once weekly.

In an embodiment, the dose of a tirzepatide composition is administered about once weekly. In an embodiment, the dose of a tirzepatide composition is administered once every seven days.

In an embodiment, there is provided a method of treating diabetes comprising administering to a human in need thereof an effective dose of one of the above-described compositions.

In an embodiment, there is provided a method of treating obesity comprising administering to a human in need thereof an effective dose of one of the above-described compositions comprising 40 mg/mL tirzepatide, or a pharmaceutically acceptable salt thereof. In an embodiment, there is provided a method of treating obesity comprising administering to a human in need thereof an effective dose of one of the above-described compositions comprising 50 mg/mL tirzepatide, or a pharmaceutically acceptable salt thereof. In an embodiment, there is provided a method of providing therapeutic weight loss comprising administering to a human in need thereof an effective dose of one of the above-described compositions. In an embodiment, there is provided a method of providing therapeutic weight loss comprising administering to a human in need thereof an effective dose of a composition comprising 40 mg/mL tirzepatide, or a pharmaceutically acceptable salt thereof. In an embodiment, tirzepatide is a free base.

In an embodiment, is a method for additional weight management in a subject in need thereof wherein the subject has a BMI≥27 kg/m² and at least one weight-related comorbidity; comprising:
administer to said subject 2.5 mg once weekly tirzepatide dose; or a pharmaceutically acceptable salt thereof, for at least 4 weeks;
after 4 weeks, increase the once weekly tirzepatide, or a pharmaceutically acceptable salt thereof, dose by administering a 5 mg dose;
increase the dose in 2.5 mg increments after at least 4 weeks at the current dose;
if additional weight management is required after at least 4 weeks at a 15 mg once weekly tirzepatide, or a pharmaceutically acceptable salt thereof;
increase the dose in 5.0 mg increments after at least 4 weeks at the current dose; and
the maximum dosage is 25 mg subcutaneous once weekly tirzepatide, or a pharmaceutically acceptable salt thereof.

In an embodiment, is a method for additional weight management in a subject in need thereof wherein the subject has a BMI≥27 kg/m² and at least one weight-related comorbidity; comprising:
administer to said subject 2.5 mg once weekly tirzepatide dose; or a pharmaceutically acceptable salt thereof, for at least 4 weeks;
after 4 weeks, increase the once weekly dose of tirzepatide, or a pharmaceutically acceptable salt thereof, dose by administering a 5 mg dose;
increase the dose in 2.5 mg increments after at least 4 weeks at the current dose;
if additional weight management is required after at least 4 weeks at a 15 mg once weekly tirzepatide, or a pharmaceutically acceptable salt thereof;
increase the dose in 2.5 mg increments after at least 4 weeks at the current dose, by administering a 17.5 mg once weekly tirzepatide dose for at least 4 weeks;
increase the dose in 2.5 mg increments after at least 4 weeks at the current dose if additional weight management is required, and
the maximum dosage is 25 mg subcutaneous once weekly tirzepatide, or a pharmaceutically acceptable salt thereof.

In an embodiment, is a method for additional weight management in a subject in need thereof wherein the subject has a BMI≥30 kg/m²; comprising:
administer to said subject 2.5 mg once weekly tirzepatide dose; or a pharmaceutically acceptable salt thereof, for at least 4 weeks;
after 4 weeks, increase the once weekly dose of tirzepatide, or a pharmaceutically acceptable salt thereof, dose by administering a 5 mg dose;
increase the dose in 2.5 mg increments after at least 4 weeks at the current dose; if additional weight management is required after at least 4 weeks at a 15 mg once weekly tirzepatide, or a pharmaceutically acceptable salt thereof;
increase the dose in 2.5 mg increments after at least 4 weeks at the current dose, by administering a 17.5 mg once weekly tirzepatide dose for at least 4 weeks;
increase the dose in 2.5 mg increments after at least 4 weeks at the current dose if additional weight management is required, and
the maximum dosage is 25 mg subcutaneous once weekly tirzepatide, or a pharmaceutically acceptable salt thereof.

In an embodiment, is a method for additional weight management in a subject in need thereof with obesity; comprising:
administer to said subject a 2.5 mg once weekly tirzepatide dose;
after 4 weeks, increase the dose by administering 5 mg once weekly tirzepatide;
increase the dose in 2.5 mg increments after at least 4 weeks at the current dose;
after at least 4 weeks at a 15 mg once weekly tirzepatide dose;
increase the dose in 5.0 mg increments after at least 4 weeks at the current dose; and
the maximum dosage is 25 mg subcutaneous once weekly tirzepatide.

In an embodiment, is a method for additional weight management in a subject in need thereof with a BMI≥27 kg/m² and at least one weight-related comorbidity; comprising:
administer to said subject 2.5 mg once weekly tirzepatide dose, or a pharmaceutically acceptable salt thereof;
after 4 weeks, increase the once weekly tirzepatide dose, or pharmaceutically acceptable salt thereof to 5 mg;
increase the dose in 2.5 mg increments after at least 4 weeks at the current dose;
after at least 4 weeks at a 15 mg once weekly tirzepatide, or pharmaceutically acceptable salt thereof dose;
increase the dose in 5.0 mg increments after at least 4 weeks at the current dose; and
the maximum dosage is 25 mg subcutaneous once weekly tirzepatide, or a pharmaceutically acceptable salt thereof.

In an embodiment, there is provided a method of providing therapeutic weight loss comprising administering to a human in need thereof an effective amount of a compositions comprising 40 mg/mL tirzepatide, or a pharmaceutically acceptable salt thereof. In an embodiment, there is provided a method of providing therapeutic weight loss comprising administering to a human in need thereof an effective amount of a composition comprising 50 mg/mL tirzepatide, or a pharmaceutically acceptable salt thereof. In an embodiment, there is provided a method of providing therapeutic weight loss comprising administering to a human in need thereof an effective amount of a composition comprising 60 mg/mL tirzepatide, or a pharmaceutically acceptable salt thereof. In an embodiment, tirzepatide, or a pharmaceutically acceptable salt thereof, is a tirzepatide free base.

In an embodiment, is a method for treating obesity in a subject with an initial body mass index (BMI) of ≥30 kg/m², comprising administering 40 mg/mL tirzepatide, or a pharmaceutically acceptable salt thereof. In an embodiment, is a method for treating obesity in a subject with an initial body mass index (BMI) of ≥30 kg/m², comprising administering 50 mg/mL tirzepatide, or a pharmaceutically acceptable salt thereof. In an embodiment, is a method for treating overweight subject with an initial BMI of ≥27 kg/m², wherein the overweight subject with a BMI≥27 kg/m² has at least one weight-related comorbid condition. In an embodiment a weight-related comorbid condition is at least one condition selected from the group consisting of hypertension, type 2 diabetes mellitus, and dyslipidemia. In an embodiment, is a method for treating obesity in a subject with an initial body mass index (BMI) of ≥30 kg/m², wherein tirzepatide is a free base form. In an embodiment, is a method for treating obesity in a subject with an initial body mass index (BMI) of ≥27 kg/m², wherein tirzepatide is a free base form.

In an embodiment is a method for treating a pediatric patient with an initial body mass index at the 95 percentile or greater standardized for age and sex, comprising administering an effective amount of tirzepatide, or a pharmaceutically acceptable salt thereof, once weekly. In an embodiment is a method for treating a pediatric patient aged 12 years or older with an initial body mass index at the 95 percentile or greater standardized for age and sex, comprising administering an effective amount of tirzepatide, or a pharmaceutically acceptable salt thereof, once weekly. In an embodiment is a method for treating a pediatric subject with an initial body mass index at the 95 percentile or greater standardized for age and sex, comprising administering 2.5 mg/mL tirzepatide, or a pharmaceutically acceptable salt thereof, for at least four weeks. In an embodiment, is a method for treating a pediatric subject with an initial body mass index at the 95 percentile or greater standardized for age and sex, comprising administering 1.25 mg tirzepatide, or a pharmaceutically acceptable salt thereof. In an embodiment is a method for chronic weight management for a pediatric subject, wherein the subject has an initial BMI≥85 percentile or greater, standardized for age and sex, with at least one weight related comorbidity. In an embodiment is a method for treating a pediatric patient, wherein tirzepatide is a free base form.

In an embodiment, a subject with an initial BMI≥30 kg/m² achieves weight loss of at least 15%. In an embodiment, a subject with an initial BMI≥30 kg/m² achieves weight loss of at least 17%. In an embodiment, a subject with an initial BMI≥30 kg/m² achieves weight loss of at least 20%.

In an embodiment, a subject with type 2 diabetes and an initial BMI≥27 kg/m² achieves weight loss of at least 15%. In an embodiment, a subject with type 2 diabetes and an initial BMI≥27 kg/m² achieves weight loss of at least 17%. In an embodiment, a subject with type 2 diabetes and an initial BMI≥27 kg/m² achieves weight loss of at least 20%.

In an embodiment, a subject with a weight related comorbidity and an initial BMI≥27 kg/m² achieves weight loss of at least 15%. In an embodiment, a subject with a weight related comorbidity and an initial BMI≥27 kg/m² achieves weight loss of at least 17%. In an embodiment, a subject with a weight related comorbidity and an initial BMI≥27 kg/m² achieves weight loss of at least 20%.

In an embodiment, is a method for treating obesity in a subject with an initial body mass index (BMI) of ≥30 kg/m², comprising administering 40 mg/mL tirzepatide, or a pharmaceutically acceptable salt thereof, wherein the subject achieves at least 15% weight loss. In an embodiment, is a method for treating obesity in a subject with an initial body mass index (BMI) of ≥30 kg/m², comprising administering 50 mg/mL tirzepatide, or a pharmaceutically acceptable salt thereof, wherein the subject achieves at least 15% weight loss. In an embodiment, is a method for treating obesity in a subject with an initial body mass index (BMI) of ≥30 kg/m², comprising administering 40 mg/mL tirzepatide, or a pharmaceutically acceptable salt thereof, wherein the subject achieves at least 20% weight loss. In an embodiment, is a method for treating obesity in a subject with an initial body mass index (BMI) of ≥30 kg/m², comprising administering 50 mg/mL tirzepatide, or a pharmaceutically acceptable salt thereof, wherein the subject achieves at least 20% weight loss. In an embodiment, is a method for treating overweight subject with type 2 diabetes and an initial BMI of ≥27 kg/m², comprising administering 40 mg/mL tirzepatide, or a pharmaceutically acceptable salt thereof, wherein the overweight subject with an initial BMI≥27 kg/m² and ≤30 kg/m², achieves at least 15% weight loss. In an embodiment, is a method for treating overweight subject with type 2 diabetes and an initial BMI of ≥27 kg/m², comprising administering 50 mg/mL tirzepatide, or a pharmaceutically acceptable salt thereof, wherein the overweight subject with an initial BMI≥27 kg/m² and ≤30 kg/m² achieves at least 15% weight loss. In an embodiment, is a method for treating overweight subject with type 2 diabetes and an initial BMI of ≥27 kg/m², comprising administering 40 mg/mL tirzepatide, or a pharmaceutically acceptable salt thereof, wherein the overweight subject with an initial BMI≥27 kg/m² and ≤30 kg/m², achieves at least 20% weight loss. In an embodiment, is a method for treating overweight subject with type 2 diabetes and an initial BMI of ≥27 kg/m², comprising administering 50 mg/mL tirzepatide, or a pharmaceutically acceptable salt thereof, wherein the overweight subject with an initial BMI≥27 kg/m² and ≤30 kg/m², achieves at least 20% weight loss. In an embodiment, is a method for treating overweight subject wherein tirzepatide is a free base form. In an embodiment, is a method for treating obesity wherein tirzepatide is a free base form. In an embodiment, tirzepatide, or a pharmaceutically acceptable salt thereof is administered in a 0.5 mL composition.

In an embodiment, is a method for treating an overweight subject with a weight related comorbid condition and an initial BMI of ≥27 kg/m², comprising administering 40 mg/mL tirzepatide, or a pharmaceutically acceptable salt thereof, wherein the overweight subject with an initial BMI≥27 kg/m² and ≤30 kg/m², achieves at least 15% weight loss. In an embodiment, is a method for treating overweight subject with a weight related comorbid condition and an initial BMI of ≥27 kg/m², comprising administering 50 mg/mL tirzepatide, or a pharmaceutically acceptable salt thereof, wherein the overweight subject with an initial BMI≥27 kg/m² and ≤30 kg/m² achieves at least 15% weight loss. In an embodiment, is a method for treating overweight subject with a weight related comorbid condition and an initial BMI of ≥27 kg/m², comprising administering 40 mg/mL tirzepatide, or a pharmaceutically acceptable salt thereof, wherein the overweight subject with an initial BMI≥27 kg/m² and ≤30 kg/m², achieves at least 20% weight loss. In an embodiment, is a method for treating overweight subject with a weight related comorbid condition and an initial BMI of ≥27 kg/m², comprising administering 50 mg/mL tirzepatide, or a pharmaceutically acceptable salt thereof, wherein the overweight subject with an initial BMI≥27 kg/m² and ≤30 kg/m², achieves at least 20% weight loss.

In certain embodiments, a weight-related comorbid condition is at least one selected from the group consisting of hypertension, dyslipidemia, prediabetes, type 2 diabetes mellitus, obstructive sleep apnea and cardiovascular disease.

In certain embodiments a weight-related comorbid condition is at least one condition selected from the group consisting of hypertension, type 2 diabetes mellitus, and dyslipidemia.

In an embodiment, there is provided one of the above-described compositions for use as a medicament.

In an embodiment, there is provided one of the above-described compositions for use in the treatment of diabetes. In an embodiment, there is provided one of the above-described compositions for use in the treatment of obesity.

In an embodiment, there is provided one of the above-described compositions for use in providing therapeutic weight loss. In an embodiment, there is provided one of the above-described compositions for use in providing non-therapeutic weight loss.

According to another aspect of the present invention, there is provided an article of manufacture comprising one of the above-described compositions. In certain embodiments, the article of manufacture is a multi-use vial. In certain embodiments, the article of manufacture is a pre-filled syringe. In certain embodiments, the article of manufacture is an automatic injection apparatus (“auto-injector”). An example of an auto-injector, as contemplated herein, is presented in U.S. Pat. No. 8,734,394.

As used herein, “obesity” means a body mass index (BMI) greater than or equal to 30 kg/m². As used herein “weight management” means behaviors, techniques, and physiological processes that contribute to a person's ability to attain a maintain a healthy weight. A healthy weight for a particular patient may be determined by consultation with a health care professional; however, the World Health Organization generally defined “overweight” as an individual with a BMI that is greater than 25 kg/m². In certain embodiments, a subject in need of chronic weight management, may refer to a subject with an initial BMI greater than or equal to 27 kg/m². In certain embodiments, a subject in need of chronic weight management refers to a subject with an initial BMI greater than or equal to 27 kg/m² with at least one weight related co-morbidity. Chronic weight management treatment facilitates patient ability to achieve their healthy weight goals. In an embodiment, “chronic weight management” means, for example, a subject achieves their healthy weight goal and maintains a weight that is within their healthy weight goal range for a period of time. In an embodiment “chronic weight management” means a subject achieves their healthy weight goal and maintains a weight that is within their healthy weight goal range for at least 3 months. In an embodiment, a subject achieves their healthy weight goal and maintains a weight within their healthy weight goal range for at least 6 months. In an embodiment, “chronic weight management” means a subject achieves their healthy weight goal and maintains a weight within their healthy weight goal range for at least one year. In an embodiment, “chronic weight management” means a patient achieves their healthy weight goal and generally maintains a weight within their healthy weight goal range. As used herein “generally maintains a weight” means that most of the time, the subject's body weight is within their healthy weight goal range. In an embodiment “generally maintains a weight” means that the subject's body weight does not increase by more than 15% above their healthy goal weight during the specified time. In an embodiment, “chronic weight management” means a patient achieves their healthy weight goal and improves at least one weight-related co-morbidity measure. In an embodiment, “chronic weight management” means a patient achieves their healthy weight goal or the patient reduces their body weight and achieves their treatment goal for at least one weight-related co-morbidity. As used herein, a subject “in need of additional chronic weight management” is unable to achieve their desired weight loss goal. In certain embodiments, “in need of additional weight management” means a subject is unable to achieve their healthy weight goal using at least a 15 mg once weekly tirzepatide treatment. In certain embodiments, “in need of additional weight management” means a subject is unable to achieve their healthy weight goal and achieve their treatment goal for at least one weight related comorbidity using at least a 15 mg once weekly tirzepatide treatment. In certain embodiments, a “pediatric patient in need of chronic weight management” is a pediatric subject with an initial body mass index at the 95 percentile or greater standardized for age and sex.

In certain embodiments, a “pediatric patient in need of chronic weight management” is a pediatric subject with an initial body mass index ≥85 percent standardized for age and sex, with at least one weight related comorbidity. As used herein “pediatric” may preferably refer to a subject younger than 20 years old. In certain embodiments, “pediatric” refers to a subject older than 12 years old. In certain embodiments, “pediatric” refers to a subject younger than 18 years old. In certain embodiments, a “pediatric patient in need of chronic weight management” is a pediatric subject with an initial body mass index at the 90 percentile or greater standardized for age and sex.

As used herein, “tirzepatide” means a GIP/GLP1 co-agonist peptide as described in U.S. Pat. No. 9,474,780 and described by CAS Registry Number: 2023788-19-2. Tirzepatide is described in Example 1 of U.S. Pat. No. 9,474,780, with the following sequence:

YX₁EGTFTSDYSIX2LDKIAQKAFVQWLIAGGPSSGAPPPS

wherein X₁ is Aib; X₂ is Aib; K at position 20 is chemically modified through conjugation to the epsilon-amino group of the K side-chain with (2-[2-(2-Amino-ethoxy)-ethoxy]-acetyl)₂-(γGlu)₁-CO—(CH₂)₁₈—CO₂H; and the C-terminal amino acid is amidated as a C-terminal primary amide (SEQ ID NO: 1).

In certain embodiments of the present invention, “tirzepatide” means a compound of the formula (SEQ ID NO:1):

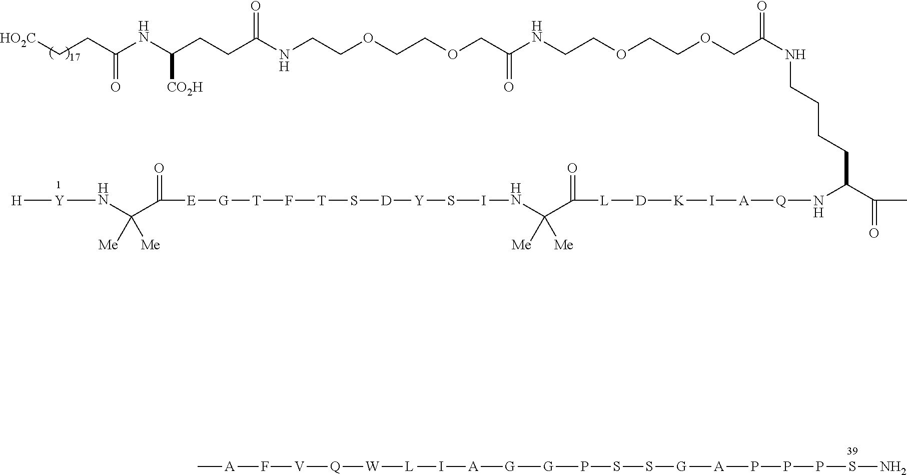

In certain embodiments of the present invention, SEQ ID NO:2) means a GIP/GLP1 co-agonist compound:

YX₁EGTFTSDYSIX₂LDKIAQKAX3VQWLIAGGPSSGAPPPS;

wherein
   X₁ is Aib;
   X₂ is Aib;
   K at position 20 is chemically modified through conjugation to the epsilon-amino group of the K side-chain with a C16-C20 fatty acid or a derivative thereof;
   X₃ is Phe;
   and the C-terminal amino acid is optionally amidated as a C-terminal primary amide; or a pharmaceutically acceptable salt thereof.

In certain embodiments as used herein SEQ ID NO:3 means a GIP/GLP1 co-agonist compound:

(SEQ ID NO: 3)
YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with a fatty acid selected from the group consisting of

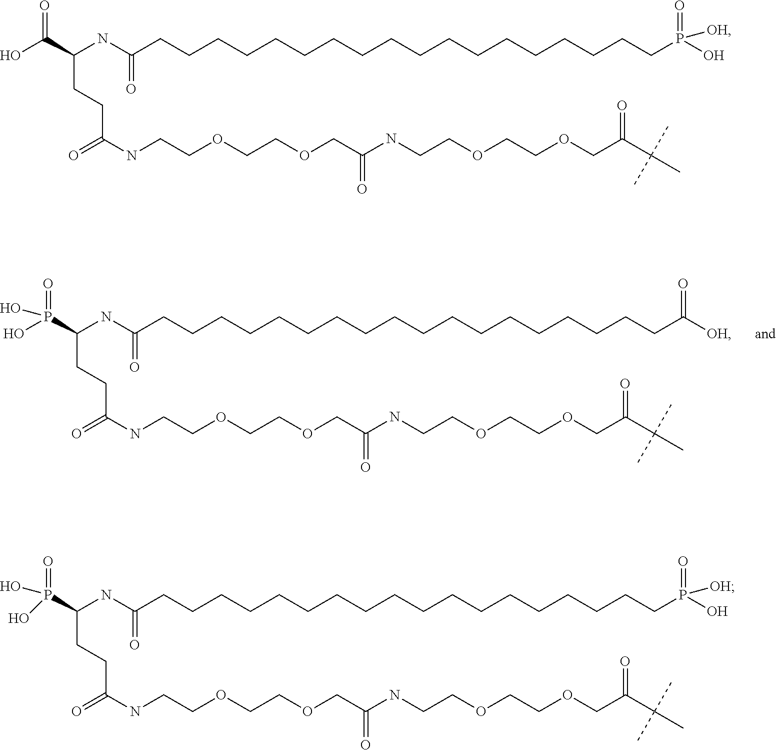

and the C-terminal amino acid is optionally amidated as a C-terminal primary amide; or a pharmaceutically acceptable salt thereof.

The term “C16-C20 fatty acid” as used herein means a diacid with between 16 and 20 carbon atoms. In an embodiment, the C16-C20 fatty acid suitable for use herein can be a saturated diacid. In an embodiment, the fatty acid is C20. In an embodiment the fatty acid is ([2-(2-Amino-ethoxy)-ethoxy]-acetyl)₂-(γGlu)ₐ-CO—(CH₂)_{b}—CO₂H wherein a is 1 to 2 and b is 10 to 18.

In certain embodiments, the C16-C20 fatty acid is

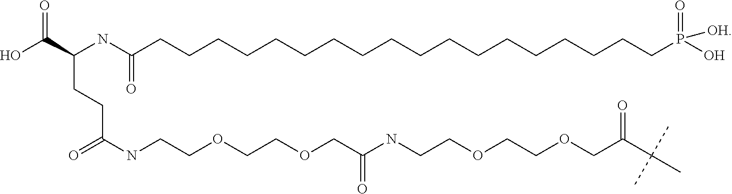

In certain embodiments, the C16-C20 fatty acid is:

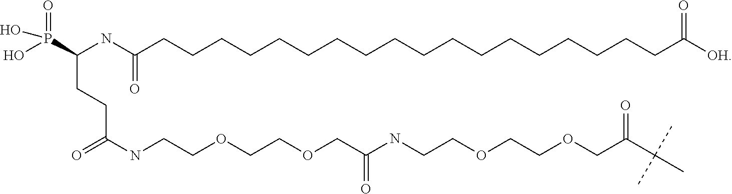

In certain embodiments, the C16-C20 fatty acid is:

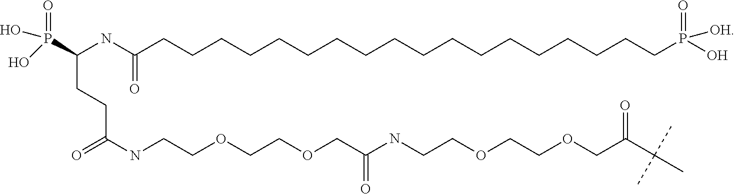

As used herein “derivative” means one atom or group of atoms is replaced with another atom or group of atoms. As used herein “derivative” may be structural analog of a C16-C20 fatty acid.

In certain embodiments, tirzepatide (SEQ ID NO:1), is preferred.

When used herein, “pharmaceutically acceptable salt” is well known to the skilled artisan. In an embodiment is a pharmaceutically acceptable salt that is a tirzepatide trifluoroacetate salt.

When used herein, the term “does not contain a surfactant” means that the composition contains no added surfactants, or contains only a de minimis quantity of an added surfactant.

The compositions of the present invention have concentrations of tirzepatide, or a pharmaceutically acceptable salt thereof, between 32 mg/mL to 55 mg/mL. The compositions of the present invention have concentrations of tirzepatide, or a pharmaceutically acceptable salt thereof, between 36 mg/mL to 55 mg/mL. In an embodiment, the concentration of tirzepatide, or a pharmaceutically acceptable salt thereof, is about 40 mg/mL to about 60 mg/mL In an embodiment, the concentration of tirzepatide is selected from the group consisting of 40 mg/mL and 60 mg/mL In an embodiment, the concentration of tirzepatide, or a pharmaceutically acceptable salt thereof, is about 40 mg/mL to about 60 mg/mL In an embodiment, the concentration of tirzepatide is selected from the group consisting of 40 mg/mL and 50 mg/mL Such compositions may be presented in a pre-filled syringe or automatic injection device. Such pre-filled syringe may be useful for administering one half milliliter of such composition per patient per dose.

The compositions of the present invention have concentrations of SEQ ID NO:2, or a pharmaceutically acceptable salt thereof, between 32 mg/mL to 55 mg/mL. The compositions of the present invention have concentrations of SEQ ID NO:2, or a pharmaceutically acceptable salt thereof, between 36 mg/mL to 55 mg/mL. In an embodiment, the concentration of SEQ ID NO:2, or a pharmaceutically acceptable salt thereof, is about 40 mg/mL to about 60 mg/mL In an embodiment, the concentration of SEQ ID NO:2 is selected from the group consisting of 40 mg/mL and 60 mg/mL In an embodiment, the concentration of SEQ ID NO:2, or a pharmaceutically acceptable salt thereof, is about 40 mg/mL to about 60 mg/mL In an embodiment, the concentration of SEQ ID NO:2 is selected from the group consisting of 40 mg/mL and 50 mg/mL.

The compositions of the present invention have concentrations of SEQ ID NO:3, or a pharmaceutically acceptable salt thereof, between 32 mg/mL to 55 mg/mL. The compositions of the present invention have concentrations of SEQ ID NO:3, or a pharmaceutically acceptable salt thereof, between 36 mg/mL to 55 mg/mL. In an embodiment, the concentration of SEQ ID NO:3, or a pharmaceutically acceptable salt thereof, is about 40 mg/mL to about 60 mg/mL In an embodiment, the concentration of SEQ ID NO:3 is selected from the group consisting of 40 mg/mL and 60 mg/mL In an embodiment, the concentration of SEQ ID NO:3, or a pharmaceutically acceptable salt thereof, is about 40 mg/mL to about 60 mg/mL In an embodiment, the concentration of SEQ ID NO:3 is selected from the group consisting of 40 mg/mL and 50 mg/mL Such compositions may be presented in a pre-filled syringe or automatic injection device. Such pre-filled syringe may be useful for administering one half milliliter of such composition per patient per dose.

The compositions of the present invention have concentrations of SEQ ID NO:4, or a pharmaceutically acceptable salt thereof, between 32 mg/mL to 55 mg/mL. The compositions of the present invention have concentrations of SEQ ID NO:4, or a pharmaceutically acceptable salt thereof, between 36 mg/mL to 55 mg/mL. In an embodiment, the concentration of SEQ ID NO:4, or a pharmaceutically acceptable salt thereof, is about 40 mg/mL to about 60 mg/mL In an embodiment, the concentration of SEQ ID NO:4 is selected from the group consisting of 40 mg/mL and 60 mg/mL In an embodiment, the concentration of SEQ ID NO:4, or a pharmaceutically acceptable salt thereof, is about 40 mg/mL to about 60 mg/mL In an embodiment, the concentration of SEQ ID NO:4 is selected from the group consisting of 40 mg/mL and 50 mg/mL Such compositions may be presented in a pre-filled syringe or automatic injection device. Such pre-filled syringe may be useful for administering one half milliliter of such composition per patient per dose.

The compositions are sterile when first produced. If provided in a multi-use vial or cartridge, an anti-microbial preservative compound or mixture of compounds that is compatible with the other components of the composition may be added at sufficient strength to meet applicable regulatory anti-microbial preservative requirements. Pharmaceutically acceptable preservatives are well-known in the art. (See, e.g., Remington: The Science and Practice of Pharmacy (D. B. Troy, Editor, 21^{st} Edition, Lippincott, Williams & Wilkins, 2006). In an embodiment, the preservative is metacresol. In an embodiment, the preservative is phenol. A composition of the invention for single use pre-filled syringe wherein the composition requires no preservative. In an embodiment, the composition does not contain a surfactant.

The pH of tirzepatide compositions of the present invention is typically 6.5 to 7.5 and it is adjusted using physiologically appropriate acids and bases, as may be required to achieve the desired pH. In an embodiment, the pH target for a tirzepatide, or pharmaceutically acceptable salt thereof, composition is between 6.7 and 7.3. Patient injection site experience is a consideration for a subcutaneously administered composition. It is desirable to select a composition associated with an acceptable patient injection site experience. For example, NaCl and citrate have been associated with painful stinging at the injection site. (Laursen, T.; Hansen, B.; Fisker, S. Pain perception after subcutaneous injections of media containing different buffers. Basic & Clinical Pharmacology & Toxicology 2006, 98, (2), 218-221.), (Fransson, J.; Espander-Jansson, Local tolerance of subcutaneous injections. Journal of Pharmacy and Pharmacology 1996, 48, (10), 1012-1015.) It is further desirable to match the tonicity (i.e., osmolality) of body fluids at the injection site as closely as possible when administering the compositions because solutions that are not approximately isotonic with body fluids can produce a painful stinging sensation when administered. It is desirable that the compositions be approximately isotonic with body fluids at the sites of injection. The present composition comprising tirzepatide, or a pharmaceutically acceptable salt thereof, NaCl, and dibasic sodium phosphate is associated acceptable patient injection site experience.

The pH of SEQ ID NO:2 compositions of the present invention is typically 6.5 to 7.5 and it is adjusted using physiologically appropriate acids and bases, as may be required to achieve the desired pH. In an embodiment, the pH target for a SEQ ID NO: 2 composition is between 6.7 and 7.3.

The pH of SEQ ID NO:3 compositions of the present invention is typically 6.5 to 7.5 and it is adjusted using physiologically appropriate acids and bases, as may be required to achieve the desired pH. In an embodiment, the pH target for a SEQ ID NO:3 composition is between 6.7 and 7.3.

The pH of SEQ ID NO:4 compositions of the present invention is typically 6.5 to 7.5 and it is adjusted using physiologically appropriate acids and bases, as may be required to achieve the desired pH. In an embodiment, the pH target for a SEQ ID NO:4 composition is between 6.7 and 7.3.

In an embodiment, the pH is adjusted using a base to facilitate dissolution in the buffer solution. The addition of an acid to the composition may be required to adjust the pH to the desired pH range. In an embodiment, NaOH is used to facilitate dissolution of tirzepatide, or a pharmaceutically acceptable salt thereof, in a buffer. In an embodiment, HCl is added to adjust the pH of the composition containing the dissolved tirzepatide, or a pharmaceutically acceptable salt thereof, to the desired pH range.

The compositions of the present invention are typically administered subcutaneously. The compositions are typically administered using a pre-filled, disposable pen, reusable pen, or automatic pen injector. The composition may be administered using a multi-use vial, single use vial, or a pump device. In an embodiment, the device is an automatic injection apparatus as claimed by U.S. Pat. No. 8,734,394.

A composition comprising 40 mg/mL tirzepatide or 50 mg/mL tirzepatide, NaCl, and dibasic sodium phosphate provides a desired shelf life stability and provides patients with an acceptable injection site experience. Likewise, a composition comprising 2.5 mg/mL tirzepatide, NaCl, and dibasic sodium phosphate provides a desired shelf life stability and provides patients with an acceptable injection site experience. As used herein, “shelf life stability” is measured under controlled conditions at about 5 degrees Celsius. A composition comprising 40 mg/mL tirzepatide, NaCl, and dibasic sodium phosphate provides acceptable in-use stability. Likewise, a composition comprising 50 mg/mL tirzepatide, NaCl, and dibasic sodium phosphate provides acceptable in-use stability. As used herein, the term “in-use stability” refers to the stability of the composition measured under controlled conditions at or about 25 degrees Celsius or at or about 30 degrees Celsius. As used herein “shelf life stability” means that degradation of tirzepatide at about 5 degrees Celsius is within the acceptable range for degradation products approved by a regulatory agency, as measured using at least one method described herein. As used herein “in-use stability” means that degradation of tirzepatide at about 25 degrees Celsius is within the acceptable range for degradation products approved by a regulatory agency, as measured using at least one method described herein. In certain embodiments, the regulatory agency is the United States Food and Drug Administration.

Although increasing the dose of a drug may, in some cases, be capable of achieving increased efficacy, increasing the dose of a drug also carries a risk of greater side effects. For example, administration of a GIP/GLP-1 Receptor agonist is known to run the risk of nausea and/or diarrhea. Thus, any increase in dose must strike a balance between sufficiently enhanced efficacy while not leading to unacceptable safety or tolerability issues.

It has been discovered that increased doses of 20 mg or 25 mg tirzepatide once weekly can be capable of providing enhanced efficacy as compared to doses up to 15 mg. In certain embodiments, a subject achieves at least 17% body weight loss. In certain embodiments, a subject achieves at least 20% body weight loss. In certain embodiments, a subject achieves at least 22% body weight loss. A once weekly 20 mg or 25 mg tirzepatide dose may be administered with acceptable safety and tolerability profiles if the dosing regimen provided herein is used for administration. Thus, the present invention provides for administering a tirzepatide dose of at least 15 mg once weekly for at least 4 weeks prior to administration of 20 mg for at least 4 weeks, resulting in an acceptable safety and tolerability profile when administering the 20 mg and 25 mg doses. In subjects for whom tirzepatide treatment is first being initiated, the dosing regimen begins with a 2.5 mg dose once weekly, for at least 4 weeks, then raising the dose to 5 mg once weekly for at four weeks, then raising the dose to 7.5 mg once weekly for at least 4 weeks, then raising the dose to 10 mg once weekly for at least 4 weeks, then raising the dose to 12.5 mg for at least 4 weeks, then raising the dose to 15 mg for at least 4 weeks, then raising the dose to 20 mg for at least 4 weeks, then, optionally, raising the dose to 25 mg once weekly. In subjects for whom tirzepatide is already being administered but in need of additional weight management, however, the dosing regimen does not require decreasing the subject's current dose. For example, in a subject who has been receiving 15 mg tirzepatide once weekly but in need of additional weight management, the regimen does not require decreasing the dose to 2.5 mg, but instead constitutes increasing the dose to 20 mg once weekly for at least 4 weeks, and then, optionally, to 25 mg. Likewise, in a subject who has been receiving 20 mg tirzepatide once weekly but in need of additional weight management, the regimen does not require decreasing the dose to 2.5 mg or 15 mg once weekly, but instead constitutes increasing the dose to 25 mg. For any of the above-described embodiments of the dosing regimen, however, the dose is preferably not increased to the next succeeding dose in the progression until the current dose has been administered for at least four weeks. In certain embodiments, a dose is increased to 20 mg tirzepatide once weekly without administration of a once weekly 17.5 mg tirzepatide dose. In certain embodiments, a dose is increased to 20 mg tirzepatide once weekly without administration of a once weekly 17.5 mg tirzepatide dose for 4 weeks prior to the first 20 mg tirzepatide once weekly dose. In certain embodiments, a dose is increased to 25 mg tirzepatide once weekly without administration of a once weekly 22.5 mg tirzepatide dose. In certain embodiments, a dose is increased to 25 mg tirzepatide once weekly without administration of a once weekly 22.5 mg tirzepatide dose for 4 weeks prior to the first 25 mg tirzepatide once weekly dose. In certain embodiments, a 25 mg tirzepatide once weekly dose is administered without administration of a 17.5 mg dose and without administration of a 22.5 mg dose. In certain embodiments, a 25 mg tirzepatide once weekly dose is administered in less than 4 weeks following the first 20 mg tirzepatide once weekly dose.

Further, increasing the tirzepatide concentration may require other modifications to the compositions, so the present invention also provides for compositions containing the increased doses of tirzepatide that will remain chemically and physically stable—and meet the current product specifications—throughout the 2 year refrigerated shelf-life and maximum in use period. In an embodiment, a composition is stable at least 3 months. In an embodiment, a composition is stable at least 6 months. In an embodiment, a composition is stable at least 3 months at 5 degrees Celsius. In an embodiment, a composition is stable at least 6 months at 30 degrees Celsius. In an embodiment, a composition is stable about 2 years at 5 degrees Celsius. In an embodiment, a composition is stable about 2 years at 30 degrees Celsius. In an embodiment, a composition is at least 90% pure active agent at the end of shelf life. In an embodiment, a composition is at least 95% pure active agent at the end of shelf life. In an embodiment, a composition is at least 90% purity tirzepatide after about 2 years at 5 degrees Celsius. In an embodiment, a composition is at least 90% purity tirzepatide after about 2 years at 30 degrees Celsius. In an embodiment, a composition is at least 95% purity tirzepatide after at least 6 months at 5 degrees Celsius. In an embodiment, a composition is at least 95% purity tirzepatide after at least 6 months at 30 degrees Celsius.

Likewise, decreasing the tirzepatide concentration may require other modifications to the compositions, so the present invention also provides for compositions containing the 2.5 mg/mL of tirzepatide that will remain chemically and physically stable—and meet the current product specifications—throughout the 2 year refrigerated shelf-life and maximum in use period.

Tirzepatide compositions studied in Phase 3 clinical trials are provided in 0.5 mL aqueous solutions comprising a dose selected from the group consisting of 2.5, 5, 7.5, 10, 12.5, and 15 mg of tirzepatide. In an embodiment, is a lower dose composition comprising 1.25 mg dose of tirzepatide. Such lower dose composition may be beneficial for a pediatric patient, or a patient in need of such dose. In an embodiment is a higher dose composition comprising a dose selected from the group consisting of 20 mg and 25 mg tirzepatide. The 1.25, 20, and 25 mg dose compositions need to provide protection against physical stress and ensure the tirzepatide remains physically stable for the duration of the product shelf-life, 2 years under refrigerated conditions, and for the maximum in use period.

As used herein “shelf life” means the time for which the material may be stored and remain suitable for use. As used herein, “suitable for use” means tirzepatide percent purity, as measured by reverse phase HPLC, is within the regulatory approval specifications for degradation products. As used herein “purity” means the percentage of active pharmaceutical ingredient remaining in a composition after a period of time. In certain embodiments, shelf life is 2 years at 30 degrees Celsius. In certain embodiments shelf life is 2 years at 5 degrees Celsius. In certain embodiments, shelf life is at least 6 months at 5 degrees Celsius. In certain embodiments, shelf life is at least 6 months at 30 degrees Celsius. In certain embodiments, tirzepatide purity of 92% as measured by reverse phase HPLC, is suitable for use. In certain embodiments, tirzepatide purity about 95%, as measured by reverse phase HPLC is suitable for use. In certain embodiments, tirzepatide purity about 90% as measured by reverse phase HPLC is suitable for use.” In certain embodiments, tirzepatide purity about 85%, as measured by reverse phase HPLC, is suitable for use. In certain embodiments, tirzepatide purity about 80% as measured by reverse phase HPLC is suitable for use.

When used herein, the term “about” refers to an amount that is within ten percent (10%) of the stated figure, wherein the intended amount may be within 10% less than the stated amount, or within 10% more than the stated amount.

When used herein, the terms “treatment,” “treat,” “treating,” and the like, are meant to include slowing or attenuating the progression of a disease, disorder, or condition. These terms also include alleviating, ameliorating, attenuating, eliminating, or reducing one or more symptoms of a disorder or condition, even if the disorder or condition is not actually eliminated and even if progression of the disorder or condition is not itself slowed or reversed.

A “subject” refers to a mammal, preferably a human with a disease, disorder or condition that would benefit from treatment with an increased dose of tirzepatide.

“Glycemic control” refers to the maintenance or reduction of a subject's HbA1c levels; “improv[ing]” glycemic control refers to reductions in HbA1c; and “in need of additional” glycemic control refers to a need for reductions in HbA1c.

“Chronic weight management” refers to a desired reduction in body weight. “Chronic weight management” may refer to treatment to reduce a subject's BMI to approach or achieve the subject's healthy weight goal. “HbA1c” refers to glycated hemoglobin levels, which develop when hemoglobin joins with glucose in the blood. HbA1c levels are a commonly used measure of glycemic control in patients with diabetes, with decreased HbA1c levels generally indicating improved glycemic control. In the context of the methods of the present invention, the methods of the present invention result in a decrease in HbA1c.

In certain embodiments of the present invention, the tirzepatide doses and dosing regimens described herein are provided for the treatment of obesity, chronic weight management and/or non-therapeutic weight loss in subjects in need thereof. In certain embodiments, the subject has a body mass index (BMI) of greater than about 25 kg/m². In certain embodiments, the subject has a body mass index (BMI) of greater than about 26 kg/m². In certain embodiments, the subjects has a body mass index (BMI) of greater than about 27 kg/m². In certain embodiments, the subject also has one or more weight-related comorbid conditions such as T2D, hypertension and/or dyslipidemia.

In certain embodiments, the doses and dosing regimens described herein are provided for the treatment of other diseases or conditions such as fatty liver disease (FLD), non-alcoholic steatohepatitis (NASH) or chronic kidney disease (CKD).

In certain embodiments, the tirzepatide doses and dosing regimens described herein are provided for the prevention and/or treatment of cognitive disorders and/or neurodegenerative disorders, such as Alzheimer's disease, Parkinson's disease, and/or multiple sclerosis.

The methods of treatment and uses described herein may be provided in simultaneous or sequential combination with other T2D treatments, including oral T2D medications such as metformin, and/or other injectable medications including rapid-acting or basal insulins.

### EXAMPLE #1—COMPOSITION CONTAINING NACL

The composition is prepared substantially as described herein. The compositions containing 2.5, 40, or 50 mg/mL of tirzepatide each contain the ingredients set forth in Table 1. Compositions that may be suitable for clinical use are provided in Table 2. Acid or base is optionally added to attain the desired pH range. Water is added quantum satis (q.s.) to one milliliter total final volume.

TABLE 1
Formulation of Tirzepatide, Phosphate, and NaCl

| | Concentration |
|---|---|
| Ingredient | (mg/mL) |
| Tirzepatide | 2.5, 40, 50 |
| dibasic | 1.34 |
| sodium phosphate* | |
| NaCl | 8.2 |

*5 mM. phosphate buffer is used

TABLE 2

| | Formulation | Formulation |
|---|---|---|
| Composition | of NaCl | of NaCl |
| Tirzepatide (mg/mL) | 40 | 50 |
| Sodium phosphate | 1.34 | 1.34 |
| dibasic 7H₂O | | |
| (mg/mL) | | |
| NaCl (mg/mL) | 8.2 | 8.2 |
| Water | q.s. to mL | q.s. to mL |

| | Low dose TZP, | Placebo |
|---|---|---|
| Ingredient | NaCl as agent | of NaCl |
| Tirzepatide (mg/mL) | 2.5 | 0 |
| Sodium phosphate | 1.34 | 1.34 |
| dibasic 7H₂O | | |
| (mg/mL) | | |
| NaCl (mg/mL)) | 8.2 | 8.2 |
| Water | q.s. to mL | q.s. to mL |

### EXAMPLE #2-COMPOSITIONS CONTAINING NACL

| | Strength | | | | | | |
|---|---|---|---|---|---|---|---|
| | 2.5 mg/ | 2.5 mg/ | 15 mg/ | 15 mg/ | 25 mg/ | 30 mg/ | 30 mg/ |
| Ingredient | 0.5 mL | 0.5 mL | 0.5 mL | 0.5 mL | 0.5 mL | 0.5 mL | 0.5 mL |
| Tirzepatide | 5 | 5 | 30 | 30 | 50 | 60 | 60 |
| (mg/mL) | | | | | | | |
| Sodium | 1.34 | 1.34 | 1.34 | 1.34 | 1.34 | 1.34 | 1.34 |
| phosphate | | | | | | | |
| dibasic | | | | | | | |
| heptahydrate | | | | | | | |
| (mg/mL) | | | | | | | |
| Sodium | 8.18 | 8.18 | 8.18 | 8.18 | 8.18 | 8.18 | 8.18 |
| Chloride | | | | | | | |
| (mg/mL) | | | | | | | |
| Water | q.s. to 1 mL | q.s. to 1 mL | q.s. to 1 mL | q.s. to 1 mL | q.s. to 1 mL | q.s. to 1 mL | q.s. to 1 mL |
| (mg/mL) | | | | | | | |
| pH target | 7.0 | 7.0 | 7.0 | 7.0 | 7.0 | 6.5 | 7.5 |

Size Exclusion Chromatography (SEC) in-Use Stability Study

This procedure is an isocratic size exclusion HPLC method with UV detection at 214 nm and is designed to determine the relative amounts of tirzepatide monomer and total aggregates. Monomer and aggregates are reported as peak area percent to the total area. The procedure is stability indicating as measured by its ability to resolve known impurities from tirzepatide. This study compares alternate compositions with the compositions of this invention, prepared as shown by Table 1.

### Shelf Life Stability Study RP-HPLC:

This procedure is a gradient reversed-phase HPLC method with UV detection at 214 nm and is designed to determine the quantity, identity, and purity of tirzepatide in the drug product. A high performance liquid chromatography (HPLC) equipped with UV detector, refrigerated autosampler, gradient analytical pump, column heater, and appropriate data collection system is used with a peptide (core shell) 2.6 μm, 4.6×250 mm column. The mobile phase A is 0.1% (v/v) trifluoracetic acid (TFA) in water. Mobile phase B is 0.1% TFA in acetonitrile (ACN). The diluent is 5 mM sodium phosphate, 140 mM NaCl at pH 7. A flow rate of 1.2 mL/min is used with a gradient. The autosampler is 5° C.±3° C. The column temperature is 60° C.±2° C. using a 10 μL injection volume (about 10 μg), with a run time of about 60 minutes. Identity is determined by matching the retention time of the main peak with that of the main peak of an external reference standard. Quantity is determined by the comparison of the main peak area with the corresponding peak in the external reference standard. Impurities and related substances are reported as peak area percent to the total peak area. The procedure measures stability by resolving known impurities from tirzepatide. A composition comprising 50 mg/mL tirzepatide with NaCl as a tonicity agent provides acceptable in-use stability for at least 6 months as shown by the greater than 89% tirzepatide purity at 6 months 30° C.

A composition comprising 50 mg/mL or 60 mg/mL tirzepatide with NaCl as a tonicity agent provides acceptable shelf life for at least 6 months at both 5° C. and 25° C. as shown by the results below. The studies demonstrate acceptable shelf life for at least 6 months at pH 6.5 to pH 7.5 as shown by studies reported below using 30 mg/0.5 with NaCl as a tonicity agent.

Comparison of High Strength (25 mg/0.5 mL) to 2.5 mg/0.5 mL and 15 mg/0.5 mL by purity by RP-HPLC (30° C.)

| | | | Initial | | |
|---|---|---|---|---|---|
| | Target | Temp | percent | 3 months | 6 months |
| Strength | pH | ° C. | purity | (% purity) | (% purity) |
| 2.5 mg/ | 7.0 | 5 | 97.5 | 97.6* | 97.6* |
| 0.5 mL | | 30 | 97.5 | 94.4 | 90.5 |
| 15 mg/ | 7.0 | 5 | 97.4 | 97.8* | 97.7 |
| 0.5 mL | | 30 | 97.4 | 94.4 | 90.0 |
| 25 mg/ | 7.0 | 5 | 97.3 | 97.3 | 96.8 |
| 0.5 mL | | 30 | 97.3 | 93.7 | 89.7 |

*RP-HPLC precision is 0.33%. 0.1% values are within error.

Comparison of High Strengths (25 mg/0.5 mL and 30 mg/0.5 mL) to 2.5 mg/0.5 mL and 15 mg/0.5 mL by purity by RP-HPLC (25° C.)

| | Target | Temp | Initial | 3 months | 6 months |
|---|---|---|---|---|---|
| Strength | pH | ° C. | (% purity) | (% purity) | (% purity) |
| 2.5 mg/ | 7.0 | 5 | 97.5 | 97.5 | 96.7 |
| 0.5 mL | | 25 | 97.5 | 96.0 | 93.1 |
| 15 mg/ | 7.0 | 5 | 97.3 | 97.4 | 96.4 |
| 0.5 mL | | 25 | 97.3 | 95.8 | 92.8 |
| 25 mg/ | 7.0 | 5 | 97.3 | 97.3 | 96.8 |
| 0.5 mL | | 25 | 97.3 | 95.5 | 93.4 |
| 30 mg/ | 6.5 | 5 | 97.3 | 97.3 | 96.6 |
| 0.5 mL | | 25 | 97.3 | 95.1 | 92.4 |
| 30 mg/ | 7.5 | 5 | 97.4 | 97.0 | 96.6 |
| 0.5 mL | | 25 | 97.4 | 94.5 | 91.3 |

### Size Exclusion Chromatography (SEC) Shelf-Life Stability Study

The size exclusion stability study methods and RP-HPLC described herein above are applied to assess compositions comprising 2.5 mg/mL tirzepatide, NaCl as a tonicity and stabilizing agent to ensure in-use stability of the lower concentration composition. The size exclusion stability study methods are applied to higher doses to assess in-use stability. Size exclusion chromatography measures tirzepatide aggregation, enabling determination of the percentage of the tirzepatide monomer. This method uses size exclusion HPLC with UV detection, and is an indicator of stability. High performance liquid chromatography is equipped with a UV detector, refrigerated autosampler, temperature control column compartment with a BEH 125 Å (3.5 μm, 7.8 mm×300 mm) size exclusion chromatography column, and data collection system. Size exclusion chromatography is completed using a reference standard that is 10 mM sodium citrate at pH 6.5. The mobile phase is 0.05% trifluoroacetic acid (TFA) in 50% acetonitrile (ACN). Column temperature is maintained at 25° C. with autosampler temperature between 2 to 8° C. Flow rate is 0.5 mL/min. The detection wavelength is 214 nm. Run time, after equilibration is about 30 minutes. Validation is completed and system suitability is checked throughout the analysis using check standard injections following blanks, and subsequent injections and at the end of the run. The % monomer is determined using the formula=(Monomer Peak Area/Total Protein Peak Area)*100. The % total high molecular weight species can be determined using the formula: (2 high molecular weight peak area (Peaks after Monomer)*100)/Total Protein Peak Area. The results below support a shelf life of at least 6 months at 5° C. with tirzepatide aggregation below 1% for all strengths studied at 5° C. The results support a shelf life of at least 6 months at 30° C. with tirzepatide aggregation at or below 1 percent for all strengths studied.

### Comparison of High Strength (25 mg/0.5 mL) to 2.5 mg/0.5 mL and 15 mg/0.5 mL by SEC HMWS (30° C.)

| | Target | Temp | Initial** | 3 months | 6 months |
|---|---|---|---|---|---|
| Strength | pH | ° C. | % | % | % |
| 2.5 mg/ | 7.0 | 5 | 0.2 | 0.2 | 0.2 |
| 0.5 mL | | 30 | 0.2 | 0.4 | 0.7 |
| 15 mg/ | 7.0 | 5 | 0.2 | 0.3 | 0.3 |
| 0.5 mL | | 30 | 0.2 | 0.6 | 1.0 |
| 25 mg/ | 7.0 | 5 | 0.2 | 0.3 | 0.4 |
| 0.5 mL | | 30 | 0.2 | 0.5 | 0.9 |

**Size exclusion chromatography percentages in table represent percent tirzepatide aggregation.

### Comparison of High Strengths (25 mg/0.5 mL and 30 mg/0.5 mL) to 2.5 mg/0.5 mL and 15 mg/0.5 mL by SEC HMWS (25° C.)

| | Target | Temp | Initial** | 3 months | 6 months |
|---|---|---|---|---|---|
| Strength | pH | ° C. | (%) | (%) | (%) |
| 2.5 mg/ | 7.0 | 5 | 0.2 | 0.2 | 0.1 |
| 0.5 mL | | 25 | 0.2 | 0.3 | 0.4 |
| 15 mg/ | 7.0 | 5 | 0.2 | 0.2 | 0.2 |
| 0.5 mL | | 25 | 0.2 | 0.4 | 0.5 |
| 25 mg/ | 7.0 | 5 | 0.2 | 0.3 | 0.4 |
| 0.5 mL | | 25 | 0.2 | 0.4 | 0.6 |
| 30 mg/ | 6.5 | 5 | 0.2 | 0.2 | 0.2 |
| 0.5 mL | | 25 | 0.2 | 0.3 | 0.5 |
| 30 mg/ | 7.5 | 5 | 0.2 | 0.2 | 0.2 |
| 0.5 mL | | 25 | 0.2 | 0.4 | 0.7 |

**Size exclusion chromatography percentages in table represent percent tirzepatide aggregation.

### EXAMPLE 3

### Clinical Studies

A randomized, double-blind, placebo-controlled study is used to evaluate the efficacy and safety of tirzepatide at 20 mg and 25 mg weekly. In this study participants have type 2 diabetes and obesity (class II obesity). This study includes 1) a screening period; 2) a dose escalation period; 3) a high dose treatment period; 4) an extension phase; and 5) post-treatment follow-up period, as described. All doses are administered using subcutaneous injection. All participants in this study must be treated with metformin for at least 3 months prior to Screening, with a minimum dose of 1500 mg/day. The prescreening metformin dose and formulation (short-acting or long-acting) should be maintained during the screening and through randomization. During the study, participants are questioned regarding any gastrointestinal symptoms.

### Period I—Screening

All screening activities must take place within a 35-day window. The purpose of screening procedures is to establish eligibility and to obtain blood samples for laboratory assessments needed to confirm eligibility. Once eligibility is confirmed, a dilated fundoscopic exam will be performed by an ophthalmologist or optometrist. Participants will be provided paper diaries and will start recording their blood glucose measurements and any hypoglycemic events. Two 7-point blood glucose measurements will be collected on 2 nonconsecutive days within 2 weeks of each visit. A 7-point blood glucose measurement consists of measurements before and 2 hours after each of 3 main meals within the same day and at bedtime.

### Period II—Dose Escalation

Following randomization, participants will be trained on self-injection of the investigational product. Date, time, and location of the first dose of study intervention will be recorded. At this visit participants will receive diabetes and weight management counseling. Beginning at randomization, all participants will receive study intervention according to their randomization arm for the duration of the 24 week escalation period. Escalation for subjects receiving tirzepatide comprise a dosing schedule initiated with 2.5 mg dose per week for 4 weeks, and increasing by 2.5 mg increments, such that subjects receive a once weekly dose that is 2.5 mg (4 weeks), 5 mg (4 weeks), 7.5 mg (4 weeks), 10 mg (4 weeks), 12.5 mg (4 weeks), 15 mg (4 weeks). Subjects randomized to placebo will receive placebo treatment during entire dose escalation period.

Discontinuation or reduction of metformin during the trial should be properly documents and recorded. Participants are considered non-compliant with the protocol if they change the dose or discontinue metformin for reasons other than severe, persistent hypoglycemia, contraindication according to country-specific label, or if short term discontinuation is in accordance with the country-specific label for metformin.

### Period III—High Dose Treatment

Participants randomly assigned to tirzepatide at visit 3 who have not discontinued study intervention will be randomly assigned to a tirzepatide maintenance dose at visit 9 (15 mg, 20 mg, or 25 mg.) Higher doses of tirzepatide (above 15 mg/week) will have additional escalation every four weeks at 5 mg increments until the randomization dose of 20 mg or 25 mg is reached. The achieve the assigned dose, all participants will be required to receive 2 injections once weekly week starting at Week 24 (that is, 20 mg dose will inject once weekly using 2-10 mg fixed dose pens, while the 25 mg dose will subsequently inject once weekly using one 10 mg fixed dose pen and one 15 mg fixed dose pen to provide the desired 25 mg total weekly dose).

Those participants will receive study intervention according to their randomization arm for the duration of the 20 week High Dose Period. Participants randomized to placebo at visit 3 will undergo a mock re-randomization at visit 9 and will continue to receive placebo for the duration of the treatment period. Participants who have permanently discontinued study intervention prior to visit 9 will not be re-randomized and will continue in the study until visit 14.

### Period IV—Primary Endpoint (Visit 14)

Participants randomized to placebo at visit 3 will not have intervention assigned and will complete the treatment period at visit 14. Those participants will complete all visit 14 procedures and proceed to the Safety Follow Up Visit at week 48. Participants who discontinued study interventions prior to visit 9 (according to randomization) will complete the treatment period at this visit. These participants will complete all visit 14 procedures and proceed to the Safety Follow Up Visit at week 48. All other participants randomized to tirzepatide will complete visit 14 procedures and continue in the study for the duration of the 36-week Extension Phase.

### Period V—Extension Phase

All participants randomly assigned to tirzepatide maintenance dose at visit 9 (second randomization) will complete all visits in the Extension Phase and complete the Safety Follow Up Visit at week 84. The extension to 80 weeks allows for 52 weeks of treatment at the highest dose achieved during dose escalation.

### Post Treatment Follow Up Visit

All posttreatment safety follow up visits will occur approximately 4 weeks following the last treatment period visit or final assessment visit. All participants are required to complete a safety follow-up visit.

Concomitant Therapy. Metoformin was chosen as a required concomitant antihyperglycemia medication. The minimum dose is at least 1500 mg/day to ensure maximum efficacy of metformin prior to adding additional therapy. Participants are directed to maintain metformin throughout the treatment period until the last dose of randomized treatment, other than special circumstances.

In certain embodiments are the following embodiments:
1. (Embodiment 1) A pharmaceutical composition comprising SEQ ID NO:2, or a pharmaceutically acceptable salt thereof; wherein the compound of SEQ ID NO: 2 concentration is selected from the group consisting of about 2.5 mg/mL, about 40 mg/mL, about 50 mg/mL, and about 60 mg/mL; NaCl; and dibasic sodium phosphate.
2. A pharmaceutical composition comprising SEQ ID NO:2, or a pharmaceutically acceptable salt thereof; wherein a compound of SEQ ID NO:2 concentration is selected from the group consisting of about 2.5 mg/mL, about 40 mg/mL, and about 50 mg/mL; NaCl; and dibasic sodium phosphate.
3. A pharmaceutical composition as embodied by Embodiment 1, wherein a compound of SEQ ID NO:2, or a pharmaceutically acceptable salt thereof, concentration is about 40 mg/mL or about 50 mg/mL.
4. A pharmaceutical composition as embodied by Embodiment 1, wherein a compound of SEQ ID NO:2, or a pharmaceutically acceptable salt thereof, concentration is about 2.5 mg/mL.
5. A pharmaceutical composition as embodied by any one of Embodiments 3 to 4, wherein dibasic sodium phosphate concentration is from about 0.7 mg/mL to about 1.5 mg/mL.
6. A pharmaceutical composition as embodied by Embodiment 5 wherein dibasic sodium phosphate concentration is about 1.34 mg/mL.
7. A pharmaceutical composition as embodied by Embodiment 6 wherein a compound of SEQ ID NO:2, or a pharmaceutically acceptable salt thereof, concentration is selected from the group consisting of about 2.5 mg/mL, about 40 mg/mL, and about 50 mg/mL.
8. A pharmaceutical composition as embodied by Embodiment 7 wherein a compound of SEQ ID NO:2, or pharmaceutically acceptable salt thereof, concentration is selected from the group consisting of 40 and 50 mg/mL.
9. A pharmaceutical composition as embodied by Embodiment 7 wherein NaCl concentration is from about 7 mg/mL to about 9 mg/mL.
10. A pharmaceutical composition as embodied by Embodiment 9 wherein NaCl concentration is from about 7.4 mg/mL to about 9.0 mg/mL.
11. A pharmaceutical composition as embodied by Embodiment 10 wherein NaCl concentration is about 8.2 mg/mL.
12. A pharmaceutical composition as embodied by Embodiment 1 wherein a compound of SEQ ID NO:2, or pharmaceutically acceptable salt thereof, concentration is selected from the group consisting of 2.5, 40, and 50 mg/mL; dibasic sodium phosphate concentration is from about 0.7 mg/mL to about 1.5 mg/mL; and NaCl concentration is from about 7 mg/mL to about 9 mg/mL.
13. A pharmaceutical composition as embodied by Embodiment 12 wherein a compound of SEQ ID NO:2, or pharmaceutically acceptable salt thereof, concentration is selected from the group consisting of 2.5, 40, and 50 mg/mL; dibasic sodium phosphate concentration is about 1.34 mg/ml; and NaCl concentration is about 8.2 mg/mL.
14. A pharmaceutical composition as embodied by Embodiment 13 wherein the composition is presented in an automatic injection apparatus.
15. A pharmaceutical composition as embodied by Embodiment 13 wherein the pH of the composition is from about 6.5 to about 7.5.
16. A pharmaceutical composition as embodied by Embodiment 15 wherein the pH is from about 6.7 to about 7.3.
17. A pharmaceutical composition as embodied by Embodiment 16 further comprising one or more preservatives.
18. A pharmaceutical composition as embodied by Embodiment 17
19, wherein the composition further comprises a preservative selected from the group consisting of metacresol and phenol.
20. A pharmaceutical composition as embodied by Embodiment 1 wherein a compound of SEQ ID NO:2, or pharmaceutically acceptable salt thereof, concentration is selected from about 2.5 mg/mL, 40 mg/mL, and 50 mg/mL; dibasic sodium phosphate is from about 0.7 to about 1.5 mg/mL; and NaCl is from about 7 mg/mL to about 9 mg/mL.
21. A pharmaceutical composition as embodied by Embodiment 20 wherein the dose of the composition is about 0.5 mL.
22. A pharmaceutical composition as embodied by Embodiment 20 wherein the composition is administered using an automatic injection apparatus.
23. A pharmaceutical composition as embodied by any one of Embodiments 1 to 22 wherein a compound is SEQ ID NO:2.
24. A method of treating diabetes comprising administering to a human in need thereof an effective dose of the pharmaceutical composition as embodied by Embodiment 20.
25. A method of treating diabetes as embodied by Embodiment 24 wherein the dose is administered once weekly.
26. A method of treating obesity comprising administering to a human in need thereof an effective dose of the pharmaceutical composition as embodied by Embodiment 20.
27. A method of treating obesity as embodied by Embodiment 26 wherein the dose is administered using an automatic injection apparatus.
28. A method of treating obesity as embodied by Embodiment 26 wherein the dose is administered once weekly.
29. A pharmaceutical composition as embodied by Embodiment 20 for use in the treatment of T2D.
30. A pharmaceutical composition as embodied by Embodiment 20 for use in the treatment of obesity.
31. A method of chronic weight management in a subject with obesity and in need of additional weight management, comprising:
   Identifying a subject with obesity and in need of additional weight management;
   Administering to said subject a once weekly dose of a compound of SEQ ID NO:2 is at least 15 mg for a minimum of four weeks; and
   After at least four weeks administering a 15 mg once weekly SEQ ID NO: 2 dose, administer a once weekly a SEQ ID NO:2 dose selected from the group consisting of 20 mg and 25 mg.
32. A method of chronic weight management as embodied by Embodiment 31, wherein the once weekly SEQ ID NO:2 dose is 20 mg.
33. A method of chronic weight management as embodied by Embodiment 31 comprising
   Administering to said subject a once weekly dose of a compound of SEQ ID NO:2 that is 20 mg for a minimum of four weeks; and
   After at least four weeks administering a 20 mg once weekly SEQ ID NO: 2 dose, administer a once weekly SEQ ID NO:2 dose that is 25 mg.
34. A method of chronic weight management in a subject in need of additional weight management, comprising:
   Identifying a subject in need of additional weight management;
   Administering to said subject a 15 mg once weekly compound of SEQ ID NO: 2 dose; and
   After at least one week, administering to said subject a once weekly SEQ ID NO:2 dose that is 20 mg for at least 2 weeks; and
   After at least 2 weeks administering to said subject a once weekly SEQ ID NO: 2 dose that is 25 mg.
35. A pharmaceutical composition comprising SEQ ID NO:3, or a pharmaceutically acceptable salt thereof; wherein the compound of SEQ ID NO:3 concentration is selected from the group consisting of about 2.5 mg/mL, about 40 mg/mL, about 50 mg/mL, and about 60 mg/mL; NaCl; and dibasic sodium phosphate.
36. A pharmaceutical composition comprising SEQ ID NO:3, or a pharmaceutically acceptable salt thereof; wherein a compound of SEQ ID NO:3 concentration is selected from the group consisting of about 2.5 mg/mL, about 40 mg/mL, and about 50 mg/mL; NaCl; and dibasic sodium phosphate.
37. A pharmaceutical composition as embodied by Embodiment 35, wherein a compound of SEQ ID NO:3, or a pharmaceutically acceptable salt thereof, concentration is about 40 mg/mL or about 50 mg/mL
38. A pharmaceutical composition as embodied by Embodiment 35, wherein a compound of SEQ ID NO:3, or a pharmaceutically acceptable salt thereof, concentration is about 2.5 mg/mL.
39. A pharmaceutical composition as embodied by any one of Embodiments 35 to 38,
   wherein dibasic sodium phosphate concentration is from about 0.7 mg/mL to about 1.5 mg/mL.
40. A pharmaceutical composition as embodied by Embodiment 39 wherein dibasic sodium phosphate concentration is about 1.34 mg/mL.
41. A pharmaceutical composition as embodied by Embodiment 40 wherein a compound of SEQ ID NO:3, or a pharmaceutically acceptable salt thereof, concentration is selected from the group consisting of about 2.5 mg/mL, about 40 mg/mL, and about 50 mg/mL.
42. A pharmaceutical composition as embodied by Embodiment 41 wherein a compound of SEQ ID NO:3, or pharmaceutically acceptable salt thereof, concentration is selected from the group consisting of 40 and 50 mg/mL.
43. A pharmaceutical composition as embodied by Embodiment 41 wherein NaCl concentration is from about 7 mg/mL to about 9 mg/mL.
44. A pharmaceutical composition as embodied by Embodiment 43 wherein NaCl concentration is from about 7.4 mg/mL to about 9.0 mg/mL.
45. A pharmaceutical composition as embodied by Embodiment 44 wherein NaCl concentration is about 8.2 mg/mL.
46. A pharmaceutical composition as embodied by Embodiment 34 wherein a compound of SEQ ID NO:3, or pharmaceutically acceptable salt thereof, concentration is selected from the group consisting of 2.5, 40, and 50 mg/mL; dibasic sodium phosphate concentration is from about 0.7 mg/mL to about 1.5 mg/mL; and NaCl concentration is from about 7 mg/mL to about 9 mg/mL.
47. A pharmaceutical composition as embodied by Embodiment 46 wherein a compound of SEQ ID NO:3, or pharmaceutically acceptable salt thereof, concentration is selected from the group consisting of 2.5, 40, and 50 mg/mL; dibasic sodium phosphate concentration is about 1.34 mg/mL; and NaCl concentration is about 8.2 mg/mL.
48. A pharmaceutical composition as embodied by Embodiment 47 wherein the composition is presented in an automatic injection apparatus.
49. A pharmaceutical composition as embodied by Embodiment 47 wherein the pH of the composition is from about 6.5 to about 7.5.
50. A pharmaceutical composition as embodied by Embodiment 49 wherein the pH is from about 6.7 to about 7.3.
51. A pharmaceutical composition as embodied by Embodiment 50 further comprising one or more preservatives.
52. A pharmaceutical composition as embodied by Embodiment 51 wherein the composition further comprises a preservative selected from the group consisting of metacresol and phenol.
53. A pharmaceutical composition as embodied by Embodiment 35 wherein a compound of SEQ ID NO:3, or pharmaceutically acceptable salt thereof, concentration is selected from about 2.5 mg/mL, 40 mg/mL, and 50 mg/mL; dibasic sodium phosphate is from about 0.7 to about 1.5 mg/mL; and NaCl is from about 7 mg/mL to about 9 mg/mL.
54. A pharmaceutical composition as embodied by Embodiment 53 wherein the dose of the composition is about 0.5 mL.
55. A pharmaceutical composition as embodied by Embodiment 53 wherein the composition is administered using an automatic injection apparatus.
56. A pharmaceutical composition as embodied by any one of Embodiments 35 to 55 wherein a compound is SEQ ID NO:3.
57. A method of treating diabetes comprising administering to a human in need thereof an effective dose of the pharmaceutical composition as embodied by Embodiment 35.
58. A method of treating diabetes as embodied by Embodiment 57 wherein the dose is administered once weekly.
59. A method of treating obesity comprising administering to a human in need thereof an effective dose of the pharmaceutical composition as embodied by Embodiment 50.
60. A method of treating obesity as embodied by Embodiment 59 wherein the dose is administered using an automatic injection apparatus.
61. A method of treating obesity as embodied by Embodiment 59 wherein the dose is administered once weekly.
62. A pharmaceutical composition as embodied by Embodiment 53 for use in the treatment of T2D.
63. A pharmaceutical composition as embodied by Embodiment 53 for use in the treatment of obesity.
64. A method of chronic weight management in a subject with obesity and in need of additional weight management, comprising:
   Identifying a subject with obesity and in need of additional weight management;
   Administering to said subject a once weekly dose of a compound of SEQ ID NO:3 is at least 15 mg for a minimum of four weeks; and
   After at least four weeks administering a 15 mg once weekly SEQ ID NO: 3 dose, administer a once weekly a SEQ ID NO:3 dose selected from the group consisting of 20 mg and 25 mg.
65. A method of chronic weight management as embodied by Embodiment 64, wherein the once weekly SEQ ID NO:3 dose is 20 mg.
66. A method of chronic weight management as embodied by Embodiment 64 comprising
   Administering to said subject a once weekly dose of a compound of SEQ ID NO:3 that is 20 mg for a minimum of four weeks; and
   After at least four weeks administering a 20 mg once weekly SEQ ID NO: 3 dose, administer a once weekly SEQ ID NO:3 dose that is 25 mg.
67. A method of chronic weight management in a subject in need of additional weight management, comprising:
   Identifying a subject in need of additional weight management;
   Administering to said subject a 15 mg once weekly compound of SEQ ID NO: 3 dose; and
   After at least one week, administering to said subject a once weekly SEQ ID NO:3 dose that is 20 mg for at least 2 weeks; and
   After at least 2 weeks administering to said subject a once weekly SEQ ID NO: 3 dose that is 25 mg.
68. A pharmaceutical composition as embodied by Embodiment 35 wherein a compound of SEQ ID NO:3, or pharmaceutically acceptable salt thereof, concentration is selected from the group consisting of 2.5, 40, and 50 mg/mL; dibasic sodium phosphate concentration is from about 0.7 mg/mL to about 1.5 mg/mL; and NaCl concentration is from about 7 mg/mL to about 9 mg/mL.
69. A pharmaceutical composition as embodied by Embodiment 46 wherein a compound of SEQ ID NO:3, or pharmaceutically acceptable salt thereof, concentration is selected from the group consisting of 2.5, 40, and 50 mg/mL; dibasic sodium phosphate concentration is about 1.34 mg/ml; and NaCl concentration is about 8.2 mg/mL.
70. A pharmaceutical composition as embodied by Embodiment 69 wherein the composition is presented in an automatic injection apparatus.
71. A pharmaceutical composition as embodied by Embodiment 35 wherein the pH of the composition is from about 6.5 to about 7.5.
72. A pharmaceutical composition as embodied by Embodiment 71 wherein the pH is from about 6.7 to about 7.3.
73. A pharmaceutical composition comprising SEQ ID NO:4, or a pharmaceutically acceptable salt thereof; wherein the compound of SEQ ID NO:4 concentration is selected from the group consisting of about 2.5 mg/mL, about 40 mg/mL, about 50 mg/mL, and about 60 mg/mL; NaCl; and dibasic sodium phosphate.
74. A pharmaceutical composition comprising SEQ ID NO:4, or a pharmaceutically acceptable salt thereof; wherein a compound of SEQ ID NO:4 concentration is selected from the group consisting of about 2.5 mg/mL, about 40 mg/mL, and about 50 mg/mL; NaCl; and dibasic sodium phosphate.
75. A pharmaceutical composition as embodied by Embodiment 73, wherein a compound of SEQ ID NO:4, or a pharmaceutically acceptable salt thereof, concentration is about 40 mg/mL or about 50 mg/mL
76. A pharmaceutical composition as embodied by Embodiment 73, wherein a compound of SEQ ID NO:4, or a pharmaceutically acceptable salt thereof, concentration is about 2.5 mg/mL.
77. A pharmaceutical composition as embodied by any one of Embodiments 73 to 76, wherein dibasic sodium phosphate concentration is from about 0.7 mg/mL to about 1.5 mg/mL.
78. A pharmaceutical composition as embodied by Embodiment 77 wherein dibasic sodium phosphate concentration is about 1.34 mg/mL.
79. A pharmaceutical composition as embodied by Embodiment 78 wherein a compound of SEQ ID NO:4, or a pharmaceutically acceptable salt thereof, concentration is selected from the group consisting of about 2.5 mg/mL, about 40 mg/mL, and about 50 mg/mL.
80. A pharmaceutical composition as embodied by Embodiment 79 wherein a compound of SEQ ID NO:4, or pharmaceutically acceptable salt thereof, concentration is selected from the group consisting of 40 and 50 mg/mL.
81. A pharmaceutical composition as embodied by Embodiment 79 wherein NaCl concentration is from about 7 mg/mL to about 9 mg/mL.
82. A pharmaceutical composition as embodied by Embodiment 81 wherein NaCl concentration is from about 7.4 mg/mL to about 9.0 mg/mL.
83. A pharmaceutical composition as embodied by Embodiment 82 wherein NaCl concentration is about 8.2 mg/mL.
84. A pharmaceutical composition as embodied by Embodiment 73 wherein a compound of SEQ ID NO:4, or pharmaceutically acceptable salt thereof, concentration is selected from the group consisting of 2.5, 40, and 50 mg/mL; dibasic sodium phosphate concentration is from about 0.7 mg/mL to about 1.5 mg/mL; and NaCl concentration is from about 7 mg/mL to about 9 mg/mL.
85. A pharmaceutical composition as embodied by Embodiment 84 wherein a compound of SEQ ID NO:4, or pharmaceutically acceptable salt thereof, concentration is selected from the group consisting of 2.5, 40, and 50 mg/mL; dibasic sodium phosphate concentration is about 1.34 mg/mL; and NaCl concentration is about 8.2 mg/mL.
86. A pharmaceutical composition as embodied by Embodiment 85 wherein the composition is presented in an automatic injection apparatus.
87. A pharmaceutical composition as embodied by Embodiment 84 wherein the pH of the composition is from about 6.5 to about 7.5.
88. A pharmaceutical composition as embodied by Embodiment 87 wherein the pH is from about 6.7 to about 7.3.
89. A pharmaceutical composition as embodied by Embodiment 85 further comprising one or more preservatives.
90. A pharmaceutical composition as embodied by Embodiment 89 wherein the composition further comprises a preservative selected from the group consisting of metacresol and phenol.
91. A pharmaceutical composition as embodied by Embodiment 73 wherein a compound of SEQ ID NO:4, or pharmaceutically acceptable salt thereof, concentration is selected from about 2.5 mg/mL, 40 mg/mL, and 50 mg/mL; dibasic sodium phosphate is from about 0.7 to about 1.5 mg/mL; and NaCl is from about 7 mg/mL to about 9 mg/mL.
92. A pharmaceutical composition as embodied by Embodiment 91 wherein the dose of the composition is about 0.5 mL.
93. A pharmaceutical composition as embodied by Embodiment 92 wherein the composition is administered using an automatic injection apparatus.
94. A pharmaceutical composition as embodied by any one of Embodiments 73 to 93 wherein a compound is SEQ ID NO:4.
95. A method of treating diabetes comprising administering to a human in need thereof an effective dose of the pharmaceutical composition as embodied by Embodiment 73.
96. A method of treating diabetes as embodied by Embodiment 95 wherein the dose is administered once weekly.
97. A method of treating obesity comprising administering to a human in need thereof an effective dose of the pharmaceutical composition as embodied by Embodiment 73.
98. A method of treating obesity as embodied by Embodiment 97 wherein the dose is administered using an automatic injection apparatus.
99. A method of treating obesity as embodied by Embodiment 97 wherein the dose is administered once weekly.
100. A pharmaceutical composition as embodied by Embodiment 73 for use in the treatment of T2D.
101. A pharmaceutical composition as embodied by Embodiment 73 for use in the treatment of obesity.
102. A method of chronic weight management in a subject with obesity and in need of additional weight management, comprising:
   Identifying a subject with obesity and in need of additional weight management;
   Administering to said subject a once weekly dose of a compound of SEQ ID NO:4 is at least 15 mg for a minimum of four weeks; and
   After at least four weeks administering a 15 mg once weekly SEQ ID NO: 4 dose, administer a once weekly a SEQ ID NO:4 dose selected from the group consisting of 20 mg and 25 mg.
103. A method of chronic weight management as embodied by Embodiment 102, wherein the once weekly SEQ ID NO:4 dose is 20 mg.
104. A method of chronic weight management as embodied by Embodiment 102 comprising
   Administering to said subject a once weekly dose of a compound of SEQ ID NO:4 that is 20 mg for a minimum of four weeks; and
   After at least four weeks administering a 20 mg once weekly SEQ ID NO: 4 dose, administer a once weekly SEQ ID NO:4 dose that is 25 mg.
105. A method of chronic weight management in a subject in need of additional weight management, comprising:
   Identifying a subject in need of additional weight management;
   Administering to said subject a 15 mg once weekly compound of SEQ ID NO: 4 dose; and
   After at least one week, administering to said subject a once weekly SEQ ID NO:4 dose that is 20 mg for at least 2 weeks; and
   After at least 2 weeks administering to said subject a once weekly SEQ ID NO: 4 dose that is 25 mg.
106. A pharmaceutical composition as embodied by Embodiment 73 wherein a compound of SEQ ID NO:4, or pharmaceutically acceptable salt thereof, concentration is selected from the group consisting of 2.5, 40, and 50 mg/mL; dibasic sodium phosphate concentration is from about 0.7 mg/mL to about 1.5 mg/mL; and NaCl concentration is from about 7 mg/mL to about 9 mg/mL.
107. A pharmaceutical composition as embodied by Embodiment 106 wherein a compound of SEQ ID NO:4, or pharmaceutically acceptable salt thereof, concentration is selected from the group consisting of 2.5, 40, and 50 mg/mL; dibasic sodium phosphate concentration is about 1.34 mg/mL; and NaCl concentration is about 8.2 mg/mL.
108. A pharmaceutical composition as embodied by Embodiment 107 wherein the composition is presented in an automatic injection apparatus.
109. A pharmaceutical composition as embodied by Embodiment 73 wherein the pH of the composition is from about 6.5 to about 7.5.
110. A pharmaceutical composition as embodied by Embodiment 109 wherein the pH is from about 6.7 to about 7.3.

### SEQUENCES

Tirzepatide
SEQ ID NO: 1
YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with (2-[2-(2-Amino-ethoxy)-ethoxy]-acetyl)₂-(γGlu)₁-CO—(CH₂)₁₈—CO₂H;
and the C-terminal amino acid is amidated as a C-terminal primary amide

SEQ ID NO: 2
YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with a C16-C20 fatty acid or a derivative thereof; and the C-terminal amino acid is optionally amidated as a C-terminal primary amide.

SEQ ID NO: 3
YX₁EGTFTSDYSIX₂LDKIAQKAFVQWLIAGGPSSGAPPPS

(SEQ ID NO: 3) wherein X₁ is Aib; X₂ is Aib; K at position 20 is chemically modified through conjugation to the epsilon-amino group of the K side-chain with a fatty acid selected from the group consisting of

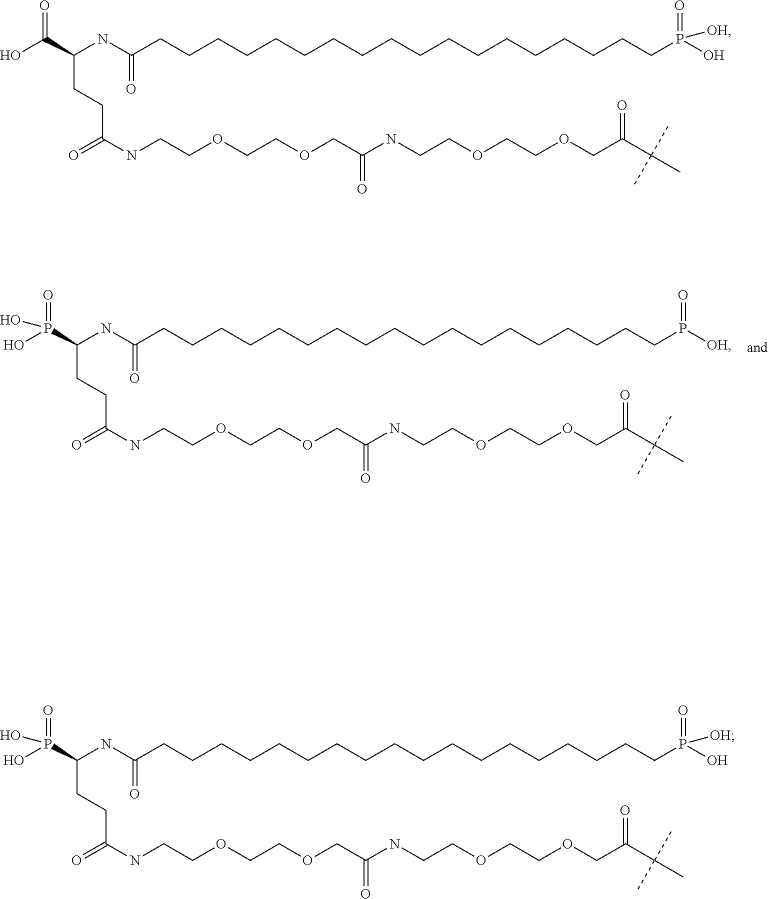

and the C-terminal amino acid is optionally amidated as a C-terminal primary amide; or a pharmaceutically acceptable salt thereof.

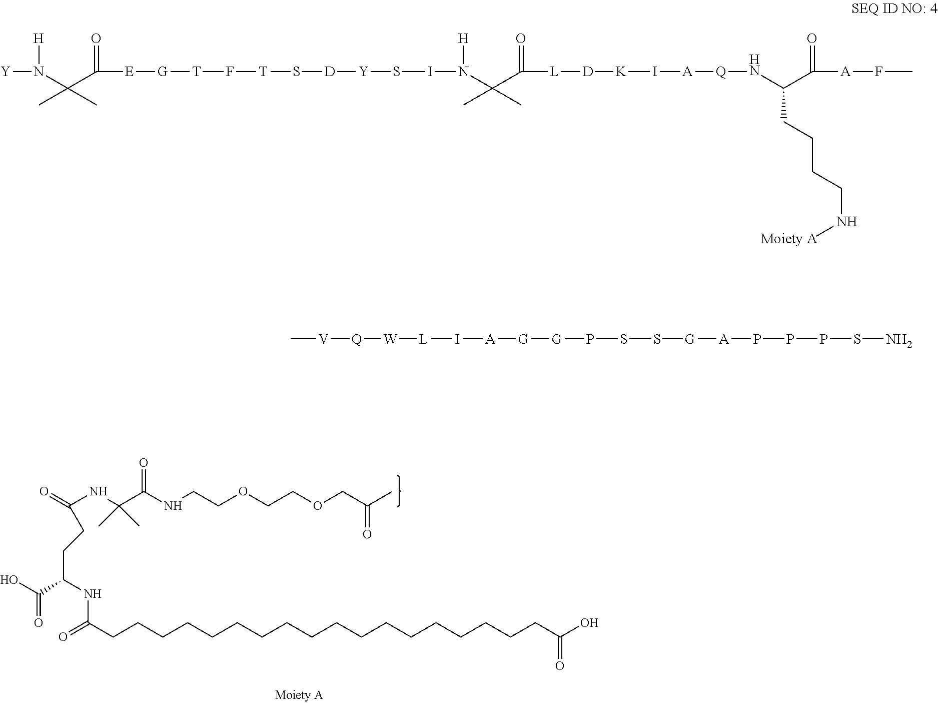

We claim:

1. A Pharmaceutical composition comprising tirzepatide, or a pharmaceutically acceptable salt thereof; NaCl; and sodium phosphate; wherein the tirzepatide, or pharmaceutically acceptable salt concentration is from about 40 mg/mL to about 60 mg/mL.

2. A pharmaceutical composition as claimed by claim 1 wherein sodium phosphate is dibasic sodium phosphate.

3. A pharmaceutical composition as claimed by claim 1 wherein the composition pH is from about 6.5 to about 7.5.

4. A pharmaceutical composition as claimed by claim 3 wherein the composition pH is from about 6.7 to about 7.3.

5. A pharmaceutical composition as claimed by claim 4 wherein tirzepatide, or a pharmaceutically acceptable salt thereof, concentration is from about 40 mg/mL to about 50 mg/mL.

6. A pharmaceutical composition as claimed by claim 5 wherein tirzepatide is a free base.

7. A pharmaceutical composition as claimed by claim 5 wherein the dibasic sodium phosphate concentration is from about 0.7 mg/mL to about 1.5 mg/mL.

8. A pharmaceutical composition as claimed by claim 7 wherein the dibasic sodium phosphate concentration is about 1.34 mg/mL.

9. A pharmaceutical composition as claimed by claim 7 wherein the NaCl concentration is from about 7 mg/mL to about 9 mg/mL.

10. A pharmaceutical composition as claimed by claim 9
wherein the NaCl concentration is from about 7.4 mg/mL to about 9.0 mg/mL.

11. A pharmaceutical composition as claimed by claim 10 wherein NaCl concentration is about 8.2 mg/mL.

12. A pharmaceutical composition as claimed by claim 10 wherein the composition shelf life stability for is at least about 6 months.

13. A pharmaceutical composition as claimed by claim 12 wherein the composition shelf life stability is for at least about 2 years.

14. A pharmaceutical composition as claimed by claim 10 wherein the composition in-use stability is at least about 3 months.

15. A pharmaceutical composition as claimed by claim 14 wherein the composition in-use stability is at least about 6 months.

16. A pharmaceutical composition comprising tirzepatide, or a pharmaceutically acceptable salt thereof; NaCl; and dibasic sodium phosphate; wherein the tirzepatide concentration is selected from the group consisting of about 2.5 mg/mL, about 40 mg/mL, about 50 mg/mL, and about 60 mg/mL.

17. A pharmaceutical composition as claimed by claim 16, wherein the composition shelf life stability is at least about 3 months.

18. A pharmaceutical composition as claimed by claim 17 wherein the composition shelf life stability is at least about 6 months.

19. A pharmaceutical composition as claimed by claim 18 wherein the composition shelf life stability is at least about 2 years.

20. (canceled)

21. A pharmaceutical composition as claimed by claim 19, wherein the tirzepatide, or a pharmaceutically acceptable salt thereof, concentration is about 40 mg/mL or about 50 mg/mL.

22. A pharmaceutical composition as claimed by claim 19, wherein the tirzepatide, or a pharmaceutically acceptable salt thereof, concentration is about 2.5 mg/mL.

23. A pharmaceutical composition as claimed by claim 19,
wherein dibasic sodium phosphate concentration is from about 0.7 mg/mL to about 1.5 mg/mL.

24. A pharmaceutical composition as claimed by claim 23 wherein dibasic sodium phosphate concentration is about 1.34 mg/mL.

25. A pharmaceutical composition as claimed by claim 19 wherein the NaCl concentration is from about 7 mg/mL to about 9 mg/mL.

26. A pharmaceutical composition as claimed by claim 25
wherein the NaCl concentration is from about 7.4 mg/mL to about 9.0 mg/mL.

27. A pharmaceutical composition as claimed by claim 26 wherein the NaCl concentration is about 8.2 mg/mL.

28. A pharmaceutical composition as claimed by claim 19 wherein tirzepatide, or pharmaceutically acceptable salt thereof, concentration is selected from the group consisting of 2.5, 40, and 50 mg/mL; dibasic sodium phosphate concentration is from about 0.7 mg/mL to about 1.5 mg/mL; and NaCl concentration is from about 7 mg/mL to about 9 mg/mL.

29. A pharmaceutical composition as claimed by claim 28 wherein tirzepatide, or pharmaceutically acceptable salt thereof, concentration is selected from the group consisting of 2.5 mg/mL, 40 mg/mL, and 50 mg/mL; dibasic sodium phosphate concentration is about 1.34 mg/mL; and NaCl concentration is about 8.2 mg/mL.

30. A pharmaceutical composition as claimed by claim 29 wherein the composition is presented in an automatic injection apparatus.

31. A pharmaceutical composition as claimed by claim 29 wherein the pH of the composition is from about 6.5 to about 7.5.

32. A pharmaceutical composition as claimed by claim 31 wherein the pH is from about 6.7 to about 7.3.

33. A pharmaceutical composition as claimed by claim 32 further comprising one or more preservatives.

34. A pharmaceutical composition as claimed by claim 33; wherein the composition further comprises a preservative selected from the group consisting of metacresol and phenol.

35. A pharmaceutical composition as claimed by claim 19 wherein tirzepatide, or pharmaceutically acceptable salt thereof, concentration is selected from 2.5 mg/mL, 40 mg/mL, and 50 mg/mL; dibasic sodium phosphate is from 0.7 mg/mL to about 1.5 mg/mL; and NaCl concentration is from 7 mg/mL to 9 mg/mL.

36. A pharmaceutical composition as claimed by claim 35 wherein tirzepatide is a free base.

37. A pharmaceutical composition as claimed by claim 35 wherein the dose of the composition is about 0.5 mL.

38. A pharmaceutical composition as claimed by claim 37 wherein the composition is administered using an automatic injection apparatus.

39. A pharmaceutical composition comprising tirzepatide, or a pharmaceutically acceptable salt thereof; wherein the tirzepatide concentration is from about 40 mg/mL to about 60 mg/mL; wherein the pH of the composition is from about 6.7 to about 7.3.

40. A pharmaceutical composition as claimed by claim 39 wherein tirzepatide is a free base.

41. A pharmaceutical composition as claimed by claim 39 wherein the tirzepatide concentration is from about 40 mg/mL to about 50 mg/mL.

42. A pharmaceutical composition as claimed by claim 41 wherein the tirzepatide concentration is selected from the group consisting of about 40 mg/mL and about 50 mg/mL

43. (canceled)

44. A pharmaceutical composition comprising tirzepatide, or a pharmaceutically acceptable salt thereof; wherein the tirzepatide concentration is selected from the group consisting of about 2.5 mg/mL, about 40 mg/mL, and about 50 mg/mL; wherein the pH of the composition is from about 6.7 to about 7.3.

45. A pharmaceutical composition as claimed by claim 19 wherein tirzepatide consists of SEQ ID NO:1.

46. A pharmaceutical composition as claimed by claim 44 for use in the treatment of T2D.

47. A pharmaceutical composition as claimed by claim 44 for use in the treatment of obesity.

48. A method for treating diabetes comprising administering to a human in need thereof an effective amount of a pharmaceutical composition as claimed by claim 44.

49. A method for treating diabetes as claimed by claim 48 wherein the effective amount is administered as a once weekly dose.

50. A method for treating obesity comprising administering to a human in need thereof an effective amount of a pharmaceutical composition as claimed by claim 44.

51. A method for treating obesity as claimed by claim 50 wherein the effective amount is administered using an automatic injection apparatus.

52. A method for treating obesity as claimed by claim 51 wherein the effective amount is administered as a once weekly dose.

53. A pharmaceutical composition as claimed by claim 44 for use in the treatment of T2D.

54. A pharmaceutical composition as claimed by claim 44 for use in the treatment of obesity.

55. A method for chronic weight management in a subject in need of additional weight management; wherein the subject has obesity, comprising:
Identifying a subject with obesity and in need of additional weight management;
Administering to said subject a once weekly dose of tirzepatide, or a pharmaceutically acceptable salt thereof, that is at least 15 mg for a minimum of four weeks; and
after at least four weeks administering the at least 15 mg once weekly dose of tirzepatide, or a pharmaceutically acceptable salt thereof, administer a once weekly dose of tirzepatide, or a pharmaceutically acceptable salt thereof, that is 5 mg greater than the last dose; wherein the maximum once weekly tirzepatide, or a pharmaceutically acceptable salt thereof, dose is 25 mg.

56. A method for chronic weight management as claimed by claim 55 wherein at least one of the once weekly doses of tirzepatide, or a pharmaceutically acceptable salt thereof, is 20 mg.

57. A method for chronic weight management as claimed by claim 55 comprising
administering to said subject a once weekly dose of tirzepatide, or a pharmaceutically acceptable salt thereof, that is 20 mg for a minimum of four weeks; and
after at least four weeks administering 20 mg once weekly tirzepatide, or a pharmaceutically acceptable salt thereof, administer a once weekly dose of tirzepatide, or a pharmaceutically acceptable salt thereof, that is 25 mg.

58. A method as claimed by claim 57 wherein tirzepatide is a free base.

59. A method for chronic weight management in a subject in need of additional weight management, comprising:
identify a subject in need of additional weight management;
administer to said subject 15 mg once weekly tirzepatide, or a pharmaceutically acceptable salt thereof; and
after at least one week, administer to said subject 20 mg once weekly tirzepatide, or a pharmaceutically acceptable salt thereof, for at least 2 weeks; and
after at least 2 weeks administering 20 mg tirzepatide, or a pharmaceutically acceptable salt thereof, once weekly;
administer 25 mg tirzepatide, or a pharmaceutically acceptable salt thereof, once weekly.

60. A method as claimed by claim 59 wherein 20 mg once weekly tirzepatide or a pharmaceutically acceptable salt thereof is administered for at least 4 weeks.

61. A method as claimed by claim 59 wherein 15 mg once weekly tirzepatide is administered for at least 4 weeks.

62. A method as claimed by claim 61 wherein tirzepatide is a free base.

63. A method for chronic weight management in a subject in need of additional weight management, comprising:
Identifying a subject in need of additional weight management;
Administer to said subject 20 mg once weekly tirzepatide, or a pharmaceutically acceptable salt thereof, for at least 2 weeks; and
after at least 2 weeks administering 20 mg once weekly tirzepatide, or a pharmaceutically acceptable salt thereof;
administer to said subject 25 mg once weekly tirzepatide, or a pharmaceutically acceptable salt thereof.

64. A method as claimed by claim 63 wherein 20 mg once weekly tirzepatide, or a pharmaceutically acceptable salt thereof, is administered without prior administration of a once weekly 17.5 mg tirzepatide dose.

65. A method as claimed by claim 64 wherein 25 mg once weekly tirzepatide, or a pharmaceutically acceptable salt, is administered without prior administration of a once weekly 22.5 mg tirzepatide dose.

66. A method as claimed by claim 64 wherein once weekly 20 mg tirzepatide, or a pharmaceutically acceptable salt thereof, is administered for a least 4 weeks.

67. A method for chronic weight management in a subject in need thereof comprising administering tirzepatide, or a pharmaceutically acceptable salt thereof, as a once weekly subcutaneous dose selected from the group consisting of 20 mg and 25 mg tirzepatide, or a pharmaceutically acceptable salt thereof.

68. (canceled)

69. A method for chronic weight management as claimed by claim 67 wherein the subject has an initial body mass index (BMI) that is less than or equal to 27 kg/m²; and wherein the subject has at least one weight-related comorbidity.

70. A method as claimed by claim 69 wherein 20 mg tirzepatide or a pharmaceutically acceptable salt thereof is administered a once weekly.

71. A method as claimed by claim 69 wherein 25 mg tirzepatide or a pharmaceutically acceptable salt thereof is administered once weekly.

72. A method as claimed by claim 71 wherein the tirzepatide, or a pharmaceutically acceptable salt thereof is administered subcutaneously.

73. A method as claimed by claim 72 wherein tirzepatide is a free base.

74. (canceled)

75. (canceled)

76. A method for additional weight management in a subject in need thereof wherein the subject has a BMI≥27 kg/m² and at least one weight-related comorbidity; comprising:
administer to said subject a 2.5 mg once weekly tirzepatide dose; or a pharmaceutically acceptable salt thereof, for at least 4 weeks;
after 4 weeks, increase the once weekly tirzepatide dose, or a pharmaceutically acceptable salt thereof, to 5 mg;
increase the dose in 2.5 mg increments after at least 4 weeks at the current dose;
if additional weight management is required after at least 4 weeks at a 15 mg once weekly tirzepatide dose, or a pharmaceutically acceptable salt thereof;
increase the dose in 5.0 mg increments after at least 4 weeks at the current dose; and
the maximum dosage is 25 mg subcutaneous once weekly tirzepatide, or a pharmaceutically acceptable salt thereof.

77. A method as claimed by claim 76 wherein the maximum dose required for weight management is 20 mg once weekly tirzepatide, or a pharmaceutically acceptable salt thereof.

78. A method as claimed by claim 77 wherein administration of 20 mg once weekly tirzepatide, or a pharmaceutically acceptable salt thereof, results in greater body weight reduction than administration of 15 mg once weekly tirzepatide, or a pharmaceutically acceptable salt thereof.

79. A method as claimed by claim 76 wherein the maximum dose required for weight management is 25 mg once weekly tirzepatide, or a pharmaceutically acceptable salt thereof.

80. A method as claimed by claim 79 wherein administration of 25 mg once weekly tirzepatide, or a pharmaceutically acceptable salt thereof, results in greater body weight reduction than administration of 15 mg once weekly tirzepatide, or a pharmaceutically acceptable salt thereof.

81. A method as claimed by claim 80 wherein the administration of a 5.0 mg increment increase after 4 weeks at the current dose of tirzepatide, or pharmaceutically acceptable salt thereof, is repeated when there is a need for additional body weight reduction.

82. A method as claimed by claim 81 wherein the administration of a 5.0 mg increment increase after 4 weeks at the current dose or tirzepatide or a pharmaceutically acceptable salt thereof, is repeated when there is a need for additional glycemic control in the subject.

83. A method as claimed by claim 82 wherein tirzepatide is a free base.

84. A method for additional weight management in a subject in need thereof wherein the subject has obesity; comprising:
administer to said subject a 2.5 mg once weekly tirzepatide dose; or a pharmaceutically acceptable salt thereof, for at least 4 weeks;
after 4 weeks, increase the once weekly tirzepatide dose, or a pharmaceutically acceptable salt thereof, to 5 mg;
increase the dose in 2.5 mg increments after at least 4 weeks at the current dose;
if additional weight management is required after at least 4 weeks at a 15 mg once weekly tirzepatide dose, or a pharmaceutically acceptable salt thereof;
increase the dose in 5.0 mg increments after at least 4 weeks at the current dose; and
the maximum dosage is 25 mg subcutaneous once weekly tirzepatide, or a pharmaceutically acceptable salt thereof.

85. A method as claimed by claim 84 wherein the maximum dose required for weight management is 20 mg once weekly tirzepatide, or a pharmaceutically acceptable salt thereof.

86. A method as claimed by claim 85 wherein administration of 20 mg once weekly tirzepatide, or a pharmaceutically acceptable salt thereof, results in greater body weight reduction than administration of 15 mg once weekly tirzepatide, or a pharmaceutically acceptable salt thereof.

87. A method as claimed by claim 86 the maximum dose required for chronic weight management is 25 mg once weekly tirzepatide, or a pharmaceutically acceptable salt thereof.

88. (canceled)

89. A method as claimed by claim 87 wherein the administration of a 5.0 mg increment increase after 4 weeks at the current dose of tirzepatide, or pharmaceutically acceptable salt thereof, is repeated when there is a need for additional body weight reduction.

90. A method as claimed by claim 89 wherein the administration of a 5.0 mg increment increase after 4 weeks at the current dose or tirzepatide or a pharmaceutically acceptable salt thereof, is repeated when there is a need for additional glycemic control in the subject.

91. A method as claimed by claim 90 wherein tirzepatide is a free base.

92. A method for additional glycemic control in a subject in need thereof, wherein the subject has type 2 diabetes; comprising:
administer to said subject a 2.5 mg once weekly tirzepatide dose; or a pharmaceutically acceptable salt thereof, for at least 4 weeks;
after 4 weeks, increase the once weekly tirzepatide dose, or a pharmaceutically acceptable salt thereof, to 5 mg;
increase the dose in 2.5 mg increments after at least 4 weeks at the current dose;
if additional glycemic control is required after at least 4 weeks at a 15 mg once weekly tirzepatide dose, or a pharmaceutically acceptable salt thereof;
increase the dose in 5.0 mg increments after at least 4 weeks at the current dose; and
the maximum dosage is 25 mg subcutaneous once weekly tirzepatide, or a pharmaceutically acceptable salt thereof.

93. A method as claimed by claim 92 wherein the maximum dose required for additional glycemic control is 20 mg once weekly tirzepatide, or a pharmaceutically acceptable salt thereof.

94. A method as claimed by claim 93 wherein administration of 20 mg once weekly tirzepatide, or a pharmaceutically acceptable salt thereof, results in greater glycemic control than administration of 15 mg once weekly tirzepatide, or a pharmaceutically acceptable salt thereof.

95. A method as claimed by claim 94 wherein the maximum dose required for glycemic control is 25 mg once weekly tirzepatide, or a pharmaceutically acceptable salt thereof.

96. A method as claimed by claim 95 wherein the administration of a 5.0 mg increment increase after 4 weeks at the current dose of tirzepatide, or pharmaceutically acceptable salt thereof, is repeated when there is a need for additional body weight reduction in the subject.

97. (canceled)

98. (canceled)

99. A pharmaceutical composition as claimed by claim 19 wherein tirzepatide comprises SEQ ID NO:1.
